(19) United States
(12) Patent Application Publication
(10) Pub. No.: US 2006/0241020 A1
(43) Pub. Date: Oct. 26, 2006

(54) Protein tyrosine phosphatase inhibitors

(75) Inventors: Rob Hooft Van Huijsduijnen, Bernex (CH); Sebastien Walchli, Oslo (NO); Fabrizio Arigoni, Geneva (CH)

Correspondence Address: FISH & NEAVE IP GROUP;ROPES & GRAY LLP, ONE INTERNATIONAL PLACE, BOSTON, MA, 02110-2624, US

(73) Assignee: Applied Research Systems ARS Holdings N.V.

(21) Appl. No.: 10/526,164

(22) PCT Filed: Aug. 20, 2003
(86) PCT No.: PCT/EP03/50385

§ 371 Date: Aug. 24, 2005

(30) Foreign Application Priority Data
Aug. 29, 2002 (EP) 02019357.9

(51) Int. Cl. A61K 38/08; C07K 7/06

(52) U.S. Cl. 514/7; 530/329

(57) ABSTRACT

The invention relates to phosphopeptides inhibiting protein tyrosine phosphatases, and their uses.

### FIELD OF THE INVENTION

The present invention is in the field of phosphatase inhibitors. More specifically, the invention relates to phosphopeptides and phosphopeptide derivatives inhibiting protein tyrosine phosphatases, and their medical uses.

### BACKGROUND OF THE INVENTION

Nearly all intracellular signaling is governed by protein phosphorylation and dephosphorylation steps. Although the role of phosphorylating enzymes, kinases, was appreciated very early on, the active role of their counterparts, phosphatases, is now rapidly gaining recognition.

The dynamic character of phosphorylation/dephosphorylation events is best appreciated when cells are treated with a generic phosphatase inhibitor such as vanadate, which results in the massive and rapid phosphorylation of many intracellular targets, and which has pleiotrophic physiological effects.

Less than 100 PTPs have been described up to now (1,2) and it is unlikely that this number will be expanded significantly, since a screen of the first draft of the Human genome (3,4) did not result in the discovery of new genes encoding for this family of proteins (Ibberson et al., submitted).

Signaling by receptor kinases and PTPs is complex. Cytokine and growth factor receptors are activated through ligand-induced dimerization, which activates the receptor kinase. Alternatively the receptor recruits intracellular kinases such as Jaks that phosphorylate the receptor (and the Jaks themselves). Both events trigger phosphorylation cascades. It is increasingly clear, however, that multiple negative feedback mechanisms exist that modulate this pathway, including SOCS (suppressors of cytokine signalling), which bind and mask receptor phosphotyrosines, PIAS (protein inhibitors of activated STATs), receptor internalisation and degradation, and dephosphorylation by PTPs. Because PTPs exist as soluble protein, as membrane-bound “receptor-”PTPs, or associated with the endoplasmic reticulum, dephosphorylation can take place while the receptor is membrane-anchored or while the receptor is being recycled

Up to now, only few PTPs are know to be connected to physiological substrates. However, the finding that PTP1B is a negative modulator of insulin and leptin signaling has spurred considerable interest in PTPs as drug targets (5-7). It was shown that PTP1B has particular substrate specificity for the phosphorylated insulin receptor (8). PTP1B has further been shown to be the major negative regulator of Insulin Receptor Tyrosine Kinase (9,10).

PTP1B has been postulated as an antitumor target because it can dephosphorylate and activate c-src (65), and is overexpressed in ovarian (66) and breast (65) carcinomas.

Another study showed that in PTP1B-deficient cells, IGF-1 (insulin-growth-like factor-1) signaling is enhanced (68). This is not too surprising, since the insulin- and IGF-1 receptor autophosphorylation domains are nearly identical, and both are probably good PTP1B substrates. One of the observed effects of IGF-1 hypersensitivity was a protection from apoptosis, which suggests PTP1B inhibitors may have use in neurodegenerative disease with a strong apoptotic component.

PTP1B, or Protein Tyrosine Phosphatase 1B (gene names PTPN1 or PTP1B, SwissProt entry P18031) is an intracellular protein. It has 435 amino acids and has a MW of 50 kD. It is expressed multiple tissues. Its C-terminal sequence predicts it is associated with the endoplasmic reticulum membrane, and this was verified experimentally.

In addition to its negative role in insulin signaling, it has been shown that Jak2 is a substrate for PTP1B. This selectivity may explain that blocking PTP1B also results in enhanced signaling through the leptin receptor.

Phosphatases are also found in the immune system. One of the earliest PTPs that was discovered, CD45, is essential for T- and B-lymphocyte antigen receptor signaling. This PTP would therefore seem a good target for immunosuppressors. The src-homology domain containing PTP SHP1 is also involved in TCR and B-cell signaling, but SHP2 is knockout phenotype (82-84) suggests it plays a much wider role.

SHP-1 (Swissprot: P29350; Gene names PTPN6, PTP1C or HCP) is a cytosolic 67.6 kD protein of 595 amino acids. It is expressed predominantly in cells of hematopoietic origin. It contains two N-terminal SH2 (src-homology-2) domains, the catalytic domain and two C-terminal autoregulatory tyrosine phosphorylation sites. It is mostly a negative regulator in pathways involving BCR, TCR, EpoR, CSF-1, lyn, syk and c-Kit. Its inhibition or genetic ablation results in an enhanced immuneresponse.

Phophatases are further involved in the development of infectious diseases. There is an interesting set of findings that pathogens exploit PTPs to increase their survival. It is by no means clear whether these intracellular or PTP-transducing microorganisms target the same pathway, but these PTPs would a priori seem good drug targets. The earliest example is the Yersinia bacterium, which encodes a PTP called YopH that is essential for virulence in vivo (35-36). A common infectious bacterium that is responsible for gastric ulcers, H. pylori, is known to transduce a protein called CagA into gastric epidermal cells on which it thrives (37); recently it was found that CagA, upon phosphorylation, activates SHP2 (38). Another example is Salmonella, which is known to transduce a PTP called SptP into its host cells (39). Other bacteria (Mycobacteria, Salmonella) are also suspected to manipulate their hosts with PTPs (35).

More indirect evidence for pathogens using PTPs comes from the observation that an established drug for the treatment of leishmaniasis, sodium stibogluconate, strongly inhibits SHP1 and, to a lesser extent, SHP2 (40). Thus, SHP1, SHP2 and microbial PTPs appear to be effective targets in infectious diseases. SHP-2 (Swissprot: Q06124; Synonyms PTP-2C, PTP-1D, SH-PTP3, SH-PTP2; Gene names PTPN11, PTP2C or SHPTP2) is a cytosolic 68 kD protein of 593 amino acids that is widely expressed. It is mostly an agonist of cytokine receptors, including GHR, leptinR (Ob), EGFR, InsR, PDGFR and intracellular activators such as NF-κB.

Vascular endothelial monolayers play an important role in inflammation. Local inflammation involves cytokine-induced upregulation of adhesion molecules such as L- and E-selectin and increased permeability of tight junctions (41) followed by neutrophil extravasation. It was recently shown that angiopoietin-1 and its endothelial receptor Tie-2 antagonize this process (42). It was also shown that endothelial-specific PTP-β (or VE-PTP for the murine ortholog) specifically de-phosphorylates the Tie-2 receptor kinase (43). This would suggest that PTP-β is a drug target in inflammation as an inhibitor of neutrophil and macrophage extravasation. PTP-β (Swissprot: P23467) is a 224 kD type I membrane protein of 1,997 amino acids that is expressed predominantly in endothelial cells.

Finally, a tyrosine phosphatase called SAP-1 (stomach cancer-associated protein tyrosine phosphatase-1) is said to be involved in cancer (44). Sap-1 (Swissprot: Q15426) is a 123 kD type I membrane protein of 1,118 amino acids that is very weakly expressed in brain, heart and stomach.

SAP-1 was cloned in 1994 as a new member of the type I transmembrane PTP family (45). The large extracellular domain consists of eight fibronectin type III-like domains. Unlike many other Receptor PTPs, Sap-1 has a single, catalytically active tyrosine phosphatase domain, and is related to GLEPP-1, PTP-β and DEP-1 (46, 47). No SAP-1 mRNA could be detected in pancreas or colon, but mRNA and protein were highly expressed in pancreatic and colorectal cancer cells. Sap-1 expression was examined by immunohistochemistry in biopsies and its overexpression was found to correlate with the progression from adenomas with mild dysplasia into adenocarcinomas (48). Overexpression studies suggest p130cas as a substrate for SAP-1 (44).

Therefore, phosphatases emerge as “druggable” targets, for which inhibitors are searched for. Such inhibitors may e.g. be small molecular weight compound inhibitors.

Many small molecular weight inhibitors for phosphatases are known. Most of the ones that are presently under development are specific for PTP1B, such as the ones reviewed by (49).

Phosphatase inhibitors may also be peptide inhibitors, or mimetics of such peptide inhibitors. Examples for peptidomimetics inhibiting PTP1B are known, such as the phosphotyrosyl mimetics described by (50), e.g. (difluorophosphonomethyl)phenylalanine (F₂Pmp). Further examples for phosphotyrosine mimetics are (difluoronaphtylmethyl)phosphonic acids, such as the ones e.g. described by (51).

An approach to Identify peptide inhibitors for phosphatases was developed by Flint et al. (12). Their strategy implicated the use of catalytically inactive mutants that are still able to interact with their substrates, but unable to dissociate. These mutants were called “trapping mutants”. Using trapping mutants, substrates from vanadate treated cells cells were analyzed (13,14). Using biochemical approaches on the phosphatases YopH and PTPα, two groups have demonstrated the preference of these PTPs for different peptides (15,16), again demonstrating the specificity of the catalytic domain.

Assays on random peptide libraries (17) or on chemicals (18) that mimic the recognition site of the pharmacological target PTP1B demonstrated a preference for acidic residues in positions −2 and −3 from the phosphorylated tyrosine and an aromatic group at position −1. More recently, one group has performed a reverse alanine scan in order to test the affinity of PTP1B for different short peptide sequences (19), and Asante-Appiah et al. (20) have tested TC-PTP on a library of synthetic peptides, changing positions one by one with all amino acids except cysteine.

Wang Peng et al. (68) have screened combinatorial libraries by mass spectrometry and identified optimal substrates for protein tyrosine phosphatase SHP-1. This approach revealed that SHP-1 prefers an acidic residue at the −2 position, with aspartic acid being slightly better than glutamic acid. At the −1 position, SHP-1 also prefers an acidic residue, although a variety of other amino acids are also tolerated. One of the SHP-1 inhibitors Wang Peng et al. generated had the sequence RNNEFpYA-NH₂, however, this peptide was classified as “class 3”, i.e. as a least preferred substrate for SHP-1.

### SUMMARY OF THE INVENTION

The invention is based on the identification of synthetic phosphopeptides that act as “ideal” substrates for five different protein tyrosine phosphatases (PTPs), namely Sap-1, PTP1B, PTP-β, SHP1 and SHP2. The phoshopeptides are e.g. useful as inhibitors of those PTPs, for which they are specific.

Since these PTPs are involved in development of various pathological conditions, the peptide inhibitors of the invention provide for new approaches for treatment or prevention of these diseases.

### BRIEF DESCRIPTION OF THE DRAWINGS

FIG. 1 shows the SPOT analysis of PTP-1B bound peptides including the YZGXY motif.

A, PTP-1B binding to peptides issued from phage display screening. 15-mer peptides containing either tyrosine(s) or phosphotyrosine(s) (Z) were synthesized on SPOT membrane. The blot was probed with radiolabeled PTP-1B DA (a trapping mutant in which the aspartic acid in the WPD motif has been mutated into Alanine). B, Sequences of the peptides that were tested on the membrane in (A). C: Valine scan of sequence 1b-4 of (B).

FIG. 2 shows the enrichment of trapped phages with PTP-β. Phages were titered after each round of panning and the ratio between the bound and unbound fraction was calculated

FIG. 3 shows PTP-Sap1 bound to peptides including the EFZG motif on SPOT. A, PTP-Sap1 binding to peptides derived from phage display. 15-mer peptides were synthesized on the membrane, with and without phosphotyrosine(s) (Z). Binding of the radiolabeled PTP-Sap1DA was revealed by autoradiography. B, Mapping of the binding site of the clone X5 by valine (underlined) scan on SPOT. Only the first tyrosine was phosphorylated, since it was the one that showed the strongest binding in panel A. Membrane was probed with the radiolabeled PTP-Sap1DA and revealed by autoradiography.

FIG. 4 shows a sequence alignment of the region surrounding R47 of PTP1B. This alignment was performed using ClustaIW sequences alignment software. The numbers correspond to catalytic domain numbering.

FIG. 5 shows PTP-Sap1R88N binding to the peptides having the sequences shown in (B) on SPOT. 15-mer peptides were synthesized on membrane, with and without phosphotyrosine(s) (Z). Binding of radiolabeled PTP-Sap1 R88N was revealed by autoradiography. Only one clone did not bind (Sm-8). The strongest signals are obtained with clones carrying T, F or I in position −1 (Clones Sm-2, Sm-11 and Sm-15, respectively).

### DETAILED DESCRIPTION OF THE INVENTION

Known technologies, such as PTP trapping mutants, phage display and SPOT were used to identify “ideal” substrates of five different protein tryrosine phosphatases, namely Sap1, PTP1B, PTP-beta, SHP1 and SHP2.

The invention therefore relates to novel, synthetic phosphopeptides that may be used as specific inhibitors of these protein tyrosine phosphatases (PTPs).

The term “phosphopeptide”, as used herein, is meant to encompass not only phosphopeptides, but also derivatives, salts, and mimetics of phosphopeptides, be it peptidomimetics or non-peptide mimetics.

In a first aspect, the invention relates to a phosphopeptide comprising an amino acid sequence having the following characteristics:

−2: E or L or V

−1: a hydrophobic amino acid, in particular I or L

0: Y

+1: G

+2: A or T or S

+3: a hydrophobic amino acid or a phenolic amino acid, in particular F or Y

+4: A or G

wherein the numbers represent the amino acid positions in the peptide and the Y in position 0 is a phosphorylated Tyrosine residue. Any peptide having these sequence characteristics may be used as a specific Sap-1 inhibitor.

It should be noted that throughout the present application, a phosphorylated Tyrosine residue may be labelled as a “Z” in order to make clear that this Tyrosine residue, which is usually abbreviated as “Y” or Tyr, is phosphorylated.

In a preferred embodiment of the invention, the phosphopeptide comprises an amino acid sequence selected from the group consisting of

| | |
|---|---|
| ELYGSYYA | (SEQ ID NO: 1) |
| EFYGAFA | (SEQ ID NO: 2) |
| EFYGAFG | (SEQ ID NO: 3) |
| AEGELYGSLYA. | (SEQ ID NO: 4) |

For the avoidance of any doubt, for all peptide sequences indicated herein the left hand side corresponds to the N-terminal side of the peptide, and the right hand side corresponds to most C-terminal side of the peptide. Equally, the first mentioned amino acid of a given peptide corresponds the N-terminal amino acid, and the last mentioned amino acid of a peptide corresponds to the C-terminal amino acid of the peptide. It is to be understood that the peptides of the invention comprise sequences indicated herein, and that thus the N-terminal amino acid of a peptide sequence indicated herein needs not to be the N-terminus of the peptide as such, and the C-terminal amino acid of a sequence indicated herein needs not to be the C-terminus of the peptide as such.

Another aspect of the invention relates to a phosphopeptide comprising an amino acid sequence having the following characteristics:

−2: E or P

−1: a hydrophobic amino acid, in particular F

0: Y

+1: G or A

+2: T

+3: a hydrophobic amino acid, in particular Y or F or I or L

+4: G or A

wherein the numbers represent the amino acid positions in the peptide and the Y in position 0 is a phosphorylated Tyrosine residue. Any peptide having these sequence characteristics may be used as a PTP1B inhibitor.

In a preferred embodiment of the invention, the phosphopeptide comprises an amino acid sequence selected from the group consisting of

| | |
|---|---|
| EFYATYG | (SEQ ID NO: 5) |
| EFYGTYG | (SEQ ID NO: 6) |
| EFYATYA | (SEQ ID NO: 7) |
| EFYGTYA. | (SEQ ID NO: 8) |

In a further aspect of the invention, the phosphopeptide comprises an amino acid sequence having the following characteristics:

−3: an acidic amino acid, in particular E or D

−2: L or E

−1: a hydrophobic amino acid, in particular L

0: Y

+1: A or G

+2: S

+3: Y or L or an acidic amino acid

+4: a phenolic amino acid, in particular Y or F.

wherein the numbers represent the amino acid positions in the peptide and the Y in position 0 is a phosphorylated Tyrosine residue. Any peptide having these sequence characteristics may be used as a PTP-β inhibitor.

In a preferred embodiment of the invention, the phosphopeptide comprises the amino acid sequence ELLYGSYY (SEQ ID NO: 9).

In yet a further aspect of the invention, the phosphopeptide comprises an amino acid sequence having the following characteristics:

−2: E or P

−1: a hydrophobic amino acid, in particular F or Y or L

0: Y

+1: A

+2: E or Q or H

+3: a hydrophobic amino acid, in particular V or I

+4: G

wherein the numbers represent the amino acid positions in the peptide and the Y in position 0 is a phosphorylated Tyrosine residue. Any peptide having these sequence characteristics may be used as a SHP1 inhibitor.

In a preferred embodiment of the invention, the phosphopeptide comprises the amino acid sequence EFYAEVG (SEQ ID NO: 10).

A further aspect of the invention relates to a phosphopeptide comprising an amino acid sequence having the following characteristics:

−2: E or F

−1: a hydrophobic amino acid, in particular a phenolic amino acid, particularly F

0: Y

+1: A

+2: E

+3: V or I

+4: G

+5: R

wherein the numbers represent the amino acid positions in the peptide and the Y in position 0 is a phosphorylated Tyrosine residue. Any peptide having these sequence characteristics may be used as a SHP-2 inhibitor.

In a preferred embodiment of the invention, the phosphopeptide comprises the amino acid sequence EFYAEVGR (SEQ ID NO: 11).

In a further preferred embodiment, the phosphopeptide of the invention comprises less than at or about 50 amino acids, or less than at or about 30 amino acids, or less than at or about 20 amino acids, or less than at or about 15 amino acids, or about 10 amino acids or about 9 amino acids or about 8 amino acids or about 7 amino acids.

The present invention also includes:

a) active mutants of any of the peptides of the invention, in which one or more amino acid residues have been added, deleted, or substituted;
b) active fractions, precursors, salts, or derivatives of (a);
c) peptide or non-peptide mimetics designed on the sequence or the structure of a peptide of the invention, or of fragments thereof.

Active mutants of the polypeptide or peptide as defined in the present invention, or nucleic acid coding therefor, include a finite set of substantially corresponding sequences as substitution peptides or polypeptides which can be routinely obtained by one of ordinary skill in the art, without undue experimentation, based on the teachings and functional features presented in the Examples.

In accordance with the present invention, preferred changes in these active mutants are commonly known as “conservative” or “safe” substitutions. Conservative amino acid substitutions are those with amino acids having sufficiently similar chemical properties, in order to preserve the structure and the biological function of the molecule. It is clear that outside the “consensus sequence” of the phosphopeptides or derivatives thereof, insertions and deletions of amino acids may be made in the remaining sequences without altering their function, particularly if the insertions or deletions only involve a few amino acids, e.g., under thirty and preferably under ten, and do not remove or displace amino acids which are critical to the functional conformation of a (poly)peptide, for example cysteine residues.

The literature provides many models on which the selection of conservative amino acids substitutions can be performed on the basis of statistical and physico-chemical studies on the sequence and/or the structure of natural polypeptides (52, 53).

Protein design experiments have shown that the use of specific subsets of amino acids can produce foldable and active proteins, helping in the classification of amino acid substitutions which can be more easily accommodated in protein structure, and which can be used to detect functional and structural homologs and paralogs (54). Preferably, the synonymous amino acid groups and more preferred synonymous groups are those defined in Table I.

TABLE I

| | | More Preferred |
|---|---|---|
| Amino Acid | Synonymous Group | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

Active mutants produced by substitutions made on the basis of these teachings, as well as active mutants wherein one or more amino acids were eliminated or added, are amongst the objects of the present invention, that is, novel or peptides having the same biological activity of a peptide of the invention, or even improved if possible.

Salts as used herein refers to both salts of carboxyl groups and to acid addition salts of amino groups of PTP inhibitor peptides, or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of the PTP binding or inhibiting activity of the invention.

Since most phosphatases exert their activity intracellularly, it is desirable to deliver the phosphopeptides of the invention through the cell membrane into the cytoplasma. This may be achieved by the route and means of delivery, which is chosen, e.g. by using liposomes. Another possibility for intracellular delivery is attaching specific moieties to the peptides, which mediate the transfer either through the lipid bilayer, or via membrane proteins, such as channel proteins, receptors, or the like.

Therefore, in a further preferred embodiment, the phosphopeptide of the invention is linked to a cell-penetrating peptide.

Cell penetrating peptides are known in the art. They may e.g. be protein-derived, such as the penetrating, which are homeodomain-derived, or derived from the HIV tat protein, or signal-sequence-based, comprising membrane translocating sequences. Cell penetrating peptides may further be synthetic and/or chimeric, such as transportan. Examples of cell penetrating peptides, and possible mechanisms of cell penetration, are reviewed e.g. in (55) or (56).

Another possibility of gaining, or enhancing cell penetration of the compounds of the invention is to introduce lipophilic characteristics. Further, such compounds may be chemically modified, derivatized, conjugated or complexed with molecules that, being transported naturally across the cell membrane, facilitate their entry or enhance their permeability across the cell membrane and into the cytoplasm. Examples of these membrane blending agents are fusogenic polypeptides, ion-channel forming polypeptides, other membrane polypeptides, and long chain fatty acids, e.g., myristic acid, palmitic acid (U.S. Pat. No. 5,149,782). These membranes blending agents insert the molecular conjugates into the lipid bilayer of cellular membranes and facilitate their entry into the cytoplasm. Other valuable methods for transmembrane delivery of molecules exploit the mechanism of receptor mediated endocytotic activity. These receptor systems include those recognizing galactose, mannose, mannose 6-phosphate, transferrin, asialoglycoprotein, transcobalamin (vitamin B 12), insulin and other peptide growth factors such as epidermal growth factor (EGF). Nutrient receptors, such as receptors for biotin and folate, can be also advantageously used to enhance transport across the cell membrane due to the location and multiplicity of biotin and folate receptors on the membrane surfaces of most cells and the associated receptor mediated transmembrane transport processes (U.S. Pat. No. 5,108,921). Thus, a complex formed between a compound to be delivered into the cytoplasm and a ligand, such as biotin or folate, can be contacted with a cell membrane bearing biotin or folate receptors to initiate the receptor mediated trans-membrane transport mechanism and thereby permit entry of the desired compound into the cell.

Modifications of the compounds of the invention to improve penetration of the blood-brain barrier would also be useful. Peptides may be altered to increase lipophilicity (e.g. by esterification to a bulky lipophilic moiety such as cholesteryl) or to supply a cleavable “targeting” moiety that enhances retention on the brain side of the barrier (57). Alternatively, the peptide may be linked to an antibody specific for the transferrin receptor, in order to exploit that receptor's role in transporting iron across the blood-brain barrier (58). Other methods of biomimetic transport and rational drug delivery in the field of transvascular drug delivery are known in the art (59).

In a further preferred embodiment, the invention provides peptide mimetics (also called peptidomimetics), or non-peptide mimetics designed on the basis of the sequence and/or the structure of a phosphopeptide of the invention. Preferably, such peptidomimetic is not the peptide RNNEFYA-NH₂, Y being a phosphorylated Tyrosine residue.

In the mimetic, the nature of peptide or polypeptide has been chemically modified at the level of amino acid side chains, of amino acid chirality, and/or of the peptide backbone. These alterations are intended to provide PTP binding and inhibiting compounds having similar or improved therapeutic, diagnostic and/or pharmacokinetic properties.

For example, when the peptide is susceptible to cleavage by peptidases following injection into the subject is a problem, replacement of a particularly sensitive peptide bond with a non-cleavable peptide mimetic can render a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. Blocking the charged N- and C-termini of the peptides would have the additional benefit of enhancing passage of the peptide through the hydrophobic cellular membrane and into the cell. Preferred alternative groups for amino acids included in peptide mimetics are those defined in Table II.

TABLE II

| Amino Acid | Synonymous Group |
|---|---|
| Ser | D-Ser, Thr, D-Thr, allo-Thr, Met, D-Met, Met(O), |
| | D-Met(O), L-Cys, D-Cys |
| Arg | D-Arg, Lys, D-Lys, homo-Arg, D-homo-Arg, Met, Ile, |
| | D-.Met, D-Ile, Orn, D-Orn |
| Leu | D-Leu, Val, D-Val, AdaA, AdaG, Leu, D-Leu, Met, D-Met |
| Pro | D-Pro, L-l-thioazolidine-4-carboxylic acid, |
| | D-or L-1-oxazolidine-4-carboxylic acid |
| Thr | D-Thr, Ser, D-Ser, allo-Thr, Met, D-Met, Met(O), |
| | D-Met(O), Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, B-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, cis-3,4, |
| | or 5-phenylproline, Bpa, D-Bpa |
| Tyr | D-Tyr, Phe, D-Phe, L-Dopa, His, D-His |
| Cys | D-Cys, S—Me-Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, |
| | D-Met, Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S—Me-Cys, Ile, D-Ile, Leu, D-Leu, Val, D-Val |

The techniques for the synthesis and the development of peptide mimetics and other non-peptide mimetics are well known in the art (60-62). For example, miniproteins and synthetic mimics capable of disrupting protein-protein interactions and inhibiting protein complex formation have been described (63). Various methodologies, for incorporating unnatural amino acids into proteins, using both in vitro and in vivo translation systems, to probe and/or improve protein structure and function are also disclosed in the literature (64).

The invention further relates to functional derivatives of the phosphopeptides of the invention. Preferably, the phosphopeptide is not the peptide RNNEFYA-NH₂, Y being a phosphorylated Tyrosine residue.

The term “derivatives” as used herein refers to derivatives, which can be prepared from the functional groups present on the lateral chains of the amino acid moieties or on the terminal N- or C-groups according to known methods. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups.

The functional derivatives of the phosphopeptides of the invention may also be conjugated to polymers in order to improve the properties of the peptide, such as the stability, half-life, bioavailability, tolerance by the human body, or immunogenicity. To achieve this goal, a functional derivative of the peptide is generated that comprises at least one moiety attached to one or more functional groups, which occur as one or more side chains on the amino acid residues. Such a functional group may e.g. be Polyethlyenglycol (PEG). PEGylation may be carried out by known methods, and is e.g. described in WO 92/13095. Further examples for PEGylation processes are disclosed e.g. in WO 02/28437, WO 99/55377, WO 99/55376 or WO 99/27897.

Therefore, in a preferred embodiment of the present invention, the phosphopeptides of the invention are PEGylated.

Method of Production of the Peptides of the Invention

The peptides of the invention may be used by any suitable method known in the art, e.g. by molecular biological, or preferably by chemical methods.

Examples of chemical synthesis technologies are solid phase synthesis and liquid phase synthesis. As a solid phase synthesis, for example, the amino acid corresponding to the C-terminus of the peptide to be synthesised is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the -amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO₂ (nitro) and Pmc (2,2,5,7,8-pentamethylchromane-6-sulphonyl) for the guanidino groups); and tBu (t-butyl) for the hydroxyl groups).

Phoshotyrosines are synthesized by incorporating F-moc phosphotyrosines during synthesis, e.g. as described in the Examples below.

After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The compounds thus obtained are then subjected to one or more steps of purification. Purification can be carried out by any one of the methods known for this purpose, i.e. any conventional procedure involving extraction, precipitation, chromatography, electrophoresis, or the like. For example, HPLC (high performance liquid chromatography) can be used. The elution can be carried using a water-acetonitrile-based solvent commonly employed for protein purification.

The invention includes purified preparations of the compounds of the invention. Purified preparations, as used herein, refers to the preparations which are at least 1%, preferably at least 5%, by dry weight of the compounds of the invention.

Medical Utility

The invention further relates to the medical uses of the phosphopeptides of the invention.

1. The phosphatases inhibited by the phosphopeptides of the invention have been described to be implied in the development of several pathologies. Therefore, the phosphopeptides, mimetics or functional derivatives of the invention, which are specific PTP inhibitors, are used as medicaments in accordance with the present invention. Preferalby, such phosphopeptide, mimetic or functional derivative is not the peptide RNNEFYA-NH₂, Y being a phosphorylated Tyrosine residue.

In a preferred embodiment, a phosphopeptide which inhibits Sap1, or a peptidomimetic, a non-peptide mimetic; or functional derivative thereof, is used for the manufacture of a medicament for treatment and/or prevention of cancer, in particular cancer of the stomach or the intestine.

In another preferred embodiment, a phosphopeptide which inhibits PTP1B, or a peptidomimetic, a non-peptide mimetic, or functional derivative thereof, is used for the manufacture of a medicament for treatment and/or prevention of diabetes and/or obesity.

PTP1B has further been shown or suggested to be implied in tumor diseases, such as e.g. ovarian or breast carcinomas. Since enhanced IGF-1 signaling was shown in PTP1B deficient cells, inhibitors of PTP1B may also have an IGF-1 like effect and may therefore be used for prevention or treatment of IGF-1 mediated diseases, such as congestive heart failure, neurodegenerative diseases, ischemic events of the brain or demyelinating diseases.

In a further preferred embodiment, a phosphopeptide inhibiting PTP1B, or a peptidomimetic, a non-peptide mimetic, or functional derivative thereof, is used as an inhibitor or suppressor of appetite.

The invention further relates to the use of a phosphopeptide inhibiting PTP-β, or a peptidomimetic, a non-peptide mimetic, or functional derivative thereof, for the manufacture of a medicament for treatment and/or prevention of inflammation. The use of such peptides, mimetics of functional derivatives for treatment and/or prevention of multiple sclerosis is particularly preferred in accordance with the present invention.

The invention further relates to the use of a phosphopeptide inhibiting PTP-β, or a peptidomimetic, a non-peptide mimetic, or functional derivative thereof, for the manufacture of a medicament for treatment and/or prevention of angiogenesis dependent diseases, such as solid cancers or cancer metastases.

The invention further relates to the use of phosphopeptides, which inhibit the phosphatases SHP1 and SHP2, or peptidomimetics, non-peptide mimetics, or functional derivative thereof, for the manufacture of a medicament for treatment and/or prevention of an infectious disease, in particular of leishmaniasis.

A method of treating a PTP mediated disease comprising administering a pharmaceutically effective amount of a phosphopeptide, a mimetic or functional derivative of the invention to a patient in need thereof is also within the present invention.

Pharmaceutical Compositions

The invention also relates to a pharmaceutical composition comprising one or more of the phosphopeptides, mimetics, or functional derivatives thereof.

Preferably, the pharmaceutical composition further comprises a pharmaceutically acceptable carrier, excipient, stabilizer, or diluent.

The active ingredients according to the invention may be administered to an individual in a variety of ways. The routes of administration include intradermal, transdermal (e.g. in slow release formulations), intramuscular, intraperitoneal, intravenous, subcutaneous, oral, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the peptide(s) according to the invention can be administered together with other components of biologically active agents such as pharmaceutically acceptable surfactants, excipients, carriers, diluents and vehicles.

The definition of “pharmaceutically acceptable” is meant to encompass any carrier, which does not interfere with effectiveness of the biological activity of the active ingredient and that is not toxic to the host to which it is administered. For example, for parenteral administration, the active peptide(s) may be formulated in a unit dosage form for injection in vehicles such as saline, dextrose solution, serum albumin and Ringer's solution.

For parenteral (e.g. intravenous, subcutaneous, intramuscular) administration, the active peptide(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active peptide(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amount of the active peptide(s) will be a function of many variables, including the type of receptor, the affinity of the substance according to the invention to its receptor, any residual cytotoxic activity exhibited thereby, the route of administration, the clinical condition of the patient.

A “therapeutically effective amount” is such that when administered, the substance according to the invention results in inhibiting a protein tyrosine phosphatase in vivo. The dosage administered, as single or multiple doses, to an individual will vary depending upon a variety of factors, including peptide pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art.

The dose of the polypeptide according to the invention required will vary from about 0.0001 to 100 mg/kg or about 0.01 to 10 mg/kg or about 0.1 to 5 mg/kg or about 1 to 3 mg/kg, although as noted above this will be subject to a great deal of therapeutic discretion.

The daily doses are usually given in divided doses or in sustained release form effective to obtain the desired results. Second or subsequent administrations can be performed at a dosage, which is the same, less than or greater than the initial or previous dose administered to the individual. A second or subsequent administration can be administered during or prior to onset of the disease.

The invention further relates to a method for the preparation of a pharmaceutical composition comprising admixing an effective amount of a peptide, a mimetic or functional derivative of the invention with a pharmaceutically acceptable carrier.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLE

### Probing Protein Tyrosine Phosphatase Substrate Specificity Using a Random, Phosphotyrosine Containing Phase Library

Overview of Experiments

A procedure was set up that allows to make phage libraries carrying random peptide sequences with phosphotyrosines. Phages were selected by immobilized, substrate-trapping GST-PTP fusion proteins. After multiple rounds of selection, individual clones were confirmed by ELISA and SPOT analysis. The sequence encoding the critical peptide displayed was subcloned and expressed in bacteria that co-express Elk kinase. The resulting protein was then used as substrate for the wild-type version of the PTP. Using this procedure consensus sequence for several PTPs was identified. Finally, the same protocol was followed using a PTP mutated on a specific residue in the catalytic domain described to be crucial for substrate recognition.

Five PTPs were challenged against a M13 Bacteriophage library wherein each phage presents a unique (phospho)peptide. The presented peptide was embedded in a rigid structure, flanked by the natural phage's protein. This same phage display technology has already been shown to be efficient in studying kinase selectivity (21-23). A peptide repertoire was used that is displayed on the protein VIII of the phage which is the major capsid protein, thus increasing the copy number of sequences presented (24). The same library had been used before to map the fyn kinase phosphorylation site (22). The use of the randomly phosphorylated library also identified a consensus sequence for the PTB domain of Shc (22). More recently a cDNA library expressed on phage has also been shown to be efficient for the identification of ligands that interact with the SH2 domain of SHP-2 (25).

In the present example, a procedure that exploits a library of M13 phages (26) that has previously been phosphorylated, was used and followed by the “trapping” of different PTPs. A consensus sequence for PTP1B was identified, which is different to the one already described (19). The validity of the sequences obtained by using the ELISA and the SPOT techniques was confirmed. The latter method was used to test individual amino acids in the identified sequences that were discovered and to map the minimal required amino acids that are recognised. Furthermore, this technique permitted a wide screen of positive clones directly from the data obtained from DNA sequencing. Following this, new rounds of panning with related PTPs (PTP-Sap1, SHP1, SHP2 and PTP-β) were performed. These phosphatases showed preference for different phages.

Material and Methods

Cloning and Purification

PTPs were cloned and purified as described previously (27). Briefly, specific primers corresponding to the beginning and the end of the catalytic domain of every PTPs were used to amplify by PCR an EST containing the region of interest. These primers were designed with an EcoRI site at the 5′end and a NotI site at the 3′ end. These two restriction sites were used to clone the catalytic domain in frame into a pGEX4TK vector (Pharmacia). For the construction of the trapping mutants, we designed internal primers that create a D to A mutation as described (27). Mutation for the R88 position within PTP-Sap1 was performed using the following internal primers: for 5′ATT GTA GCG GTT CTT GGC GT3′ and 5′ACG CCA AGA ACC GCT ACA ATA ATG TGC TGC CCT ATG ACT G3′. The clones used for SPOT analysis were subcloned into modified pGEX2TK encoding for a PKA phosphorylation site in-frame with GST. Constructs were checked by sequencing. Escherichia coli BL21 (codon +; Stratagene) were transformed, and a single colony was grown in 25 mL LB+amp+Cm at 37° C. until an OD of 0.5 was reached. Protein production was performed at 30° C. for 3 hours after the addition of IPTG at 250 μM final concentration. The bacteria were pelleted and ressuspended in lysis buffer (50 mM tris pH 8.0, 5 mM EDTA, 0.1% Triton X-100, 150 mM NaCl+a proteinase inhibitor cocktail, Complete™ (Roche Molecular Biochemicals)) and lysed by a treatment with lyzozyme (200 μg/ml final) for 1 hour on ice followed by three rounds of sonication. Supernatant of the lysate was incubated for more than 2 hours with 100 μl of a 50% solution of glutathione Sepharose beads (Pharmacia) at 4° C. Finally, beads were extensively washed and the PTPs were eluted in 50 mM Tris pH 8.0 with 10 mM glutathione. Glycerol was added to a final concentration of 20%, amount of proteins produced was determined with Bio Rad protein assay and aliquots were stocked at −20° C. until use.

PEP-GST constructs were prepared as follows: the primers used correspond to the pVIII capsid sequence of M13+two restriction sites (XhoI and NotI): 5′TAT CTC GAG TCT TTC GCT GCT GAG GGT GA3′ for the sense and 5′ATA GCG GCC GCT TGC AGG GAG TCA AAG GCC G3′ for the antisense. The DNA of the phage was directly amplified by adding 10⁹ phages to the PCR mix. After PCR using Herculase Polymerase (Stratagene), the DNA fragments (100 bp) were purified using Microcon® PCR (Millipore) and gel extraction was performed with Utrafree®-DA (Millipore). The cloning and the protein purification were finally accomplished following the same protocol as for the PTPs, with the only difference that TKB1 (Stratagene) bacteria were transformed and a single colony was grown and induced for protein production and phosphorylation exactly as described by the manufacturer. Again, all constructs were checked by DNA sequencing before protein production.

Labelling of GST-PTP for the SPOT analysis was performed as follows: 2 to 5 μg of GST fusion proteins were bound to glutathion-sepharose beads (Amersham Pharmacia Biotech) at 4° C. After washes GST fusion proteins were radiolabeled by 50 units of protein kinase from bovine heart (Sigma) in presence of 35 μCi of (γ-32P) ATP in PKA buffer (20 mM Tris pH 7.5, 100 mM NaCl, 12 mM MgCl2, 1 mM DTT) for 30 minutes on ices. After washes, fusion proteins were eluted from beads by 10 mM free glutathione in 50 mM Tris, pH 8.0 (28).

Phosphorylation of the Library

Phages (10⁹-10¹⁰) were incubated for three hours at 30° C. in the kinase buffer (25 mM Tris pH7.5, 5 mM MgCl₂, 2.5 mM MnCl₂, 2.5 mM DTT and 1 mM ATP) in a total volume of 20 μl. The crude library was first assayed on the PTPs trapping mutant, but the background did not allow to observe any amplification of the number of bound clones phosphorylated and non phosphorylated (data not shown). Therefore, the total library was phosphorylated and passed through an antiPTyr column (Upstate). The bound phages were eluted with phenylphosphate, and the clones were passed twice again in the column. The amplified Tyrosine-carrying phages were then stocked at 4° C. in TE and used for the trapping experiment (see below). Sequence analysis of the resulting phages showed no bias in the composition of the amino acids surrounding the Tyrosine and only two clones out of 30 sequenced did not carry any Tyrosine at all (results not shown). Furthermore, no sequences were found twice. We confirmed the results obtained previously ((22,25,29) and data not shown) that showed no phosphorylation of the wild type phage versus a high degree of phosphorylation of the library. This was checked following our phosphorylation protocol and using (γ-32P) ATP as a tracer.

Panning, Amplification and Sequencing of the Selected Phages

Phages (10⁸ to 10⁹) were incubated in kinase buffer (20 mM Tris pH7.5, 5 mM MgCl₂, 2.5 mM MnCl₂, 1 mM ATP, 2.5 mM DTT and with or without 3 Units of src kinase) for 3 hours at 30° C. The p60src, purified from Baculovirus, was purchased from Upstate Biotech. Gluthation-Sepharose beads were pre-coated with 3 μg of either GST or PTP-GST for 4 hours at 4° C. in a solution of Trapping Buffer (20 mM Tris pH 7.5, 150 mM NaCl and 1 mM EDTA) with 1% BSA final. The phages were first pre-cleared using GST-beads for 30 minutes at 4° C., spun down and the supernatant was incubated with the coated PTP-GST for 1.5 hours at 4° C. with a constant shaking. Finally beads were spun down and washed several times (5 times for the first panning, then 10 times) with a solution of PBS with 0.25% Tween® final. Phages were eluted from PTP-GST by an acidic treatment (glycine buffer, pH 2.7) for 10 minutes at RT with a constant shaking, beads were spun down and phages were recovered in the supernatant. One tenth volume of a 1 M Tris pH 9.0 solution was added in order to restore the pH. Phages were finally titred in both fractions (bound and unbound) and the rest of the bound fraction was used to infect XL1 MRF′ Bacteria (Stratagene) following described procedures (24). The day after, cells were scraped and phages were amplified using helper phage M13K07 (Pharmacia) using the manufacturer's procedures.

When an increase in the number of trapped clones was observed, phages where purified as single clones and colony-PCR was performed using phage primers, forward: 5′ATG AAA AAG TCT TTA GTC CTC3′ and reverse: 5′CAG CTT GAT ACC GAT AGT TGC3′. The PCR products were then purified and sequenced using the same primers. Sequences were read in both direction using SeqmanII Software.

Dephosphorylation Assay on GST-PEP Constructs and on pNPP

100 ng of PEP-GST construct was incubated with 10 ng of PTP wild type in PTP buffer (50 mM Hepes pH 7.4, 0.05% Nonidet NP-40 and 1 mM DTT). The reaction was stopped by mixing equal volume aliquots with a solution of 50 mM vanadate at different laps of time. The total mix was finally spotted on a nitrocellulose membrane using a 96 well dot-blot apparatus (Bio-Rad) and the phosphorylation state of the substrate was visualised using anti-phospho-tyrosine antibody (clone G410, Upstate).

For pNPP hydrolysis, 200 ng of PTP was incubated with varing amounts of the generic substrate (0-60 mM) in assay buffer (50 mM Tris pH 6.8 and 2 mM DTT) in a total volume of 50 μl at room temperature. Plates were read at 405 nm and kinetic parameters were determined using a non linear regression programme (Graphpad, Prism®).

ELISA

Single clones were amplified as described previously. The coating was done with GST-PTP or GST alone in PBS (2 μg per well in a 96 well plate format). Phosphorylation of the phages was performed following the described protocol (with and without 3 U of src kinase, in order to check the phospho-Tyr specificity of the recognition, thus be sure that positives were catalytic domain specific as shown in (30)) and during this process, each well was blocked with a solution of 5% fat free powder milk in PBS. Wells were washed 5 times in PBS-Tween® 0.5% and phages were incubated with the PTP-GST or GST in 100 μl Trapping Buffer (20 mM Tris pH 7.5, 150 mM NaCl and 1 mM EDTA) for 2 hours at 4° C. Wells were extensively washed and blocked for 1 hour in PBS-5% Milk. Phage presence was detected using anti-M13 monoclonal antibody (Pharmacia) as first antibody and signal was amplified with goat anti-mouse Ig-HRP (DAKO) and revealed by peroxydase activity using ABTS (Sigma) as described in Pharmacia's protocol.

SPOT Synthesis and Probing

Peptides were manually synthesized on derivatized cellulose membrane provided by Sigma-Genosys, which also provided the 20 Fmoc-amino acids active esters. Fmoc-phosphotyrosines from Novabiochem (#04-12-1156) were incorporated in presence of the coupling reagent, N,N′-Diisopropylcarbodiimide (DIC; Sigma) and hydroxybenzotriale (HOBt; Fluka; Espanel et al., submitted).

Membranes were blocked (at least 2 hours) and probed at 4° C. in Western wash buffer (10 mM Tris, pH 7.4, 0.1% Triton X-100 and 150 mM NaCl)+SPOT blocking buffer (Sigma-Genosys). After 2 hours, membranes were washed several times in Western wash buffer and were autoradiographed.

Construction of the Tyr-Biased Library:

We used the phagemid described in (1) which displays an insert of 9 amino acids at the C-termius part of the pVIII protein. We wanted to (i) remove the EF motif that corresponds to the EcoRI site on the 5′ end of the insert and (ii) add a fixed Tyrosine on the middle of the displayed sequence. The DNA insert was designed with MunI in 5′-end and a BamH1 in the 3′ end (underlined). The primer was also prolonged with the reverse sequence of pGXb (bold) primer for the filling with the polymerse. Thus, the sequence of the synthetic oligonucleotides encoding for ELXXXXYXXXXDP (X means any amino acid) is: 5′-ATA CAA TTG (NNK)₄ TAT (NNK)₃ NNG GAT CCT ACA CAT GCA GCT CCC GGA G, where n=A, T, G or C and K=G or T. The method used to construct the library was performed as described in (2, 3). Briefly, 400 pmole of the library primer and pGXb (5′-GTC TCC GGG AGC TGC ATG TG-3′) were annealed and the complementary strand was filled using Klenow DNA PoII (New England Biolabs). The DNA was then extracted by a phenol-chloroform precipitation and the product was digested by MunI and BamHI. The mix was then loaded on a 15% polyacrylamide gel with control and the band migrating at the correct size was cut and recovered (4). The insert was clones into the pC89 vector which had been previously opened with EcoRI and BamHI (1). XL1BlueMRF′ electrocompetent cells (Stratagene) were transformed with the ligation and propagated in 20 large plate (100 ml LB-agar with ampicilin and tetracycline). Cells were harvested and frozen in 2 mL aliquots.

Phages were amplified as follows: 2 ml of library bacteria were diluted in 5 liters of LB supplemented with Tet (12.5 μg/ml) and Amp (50 μg/ml), shacked until OD₆₀₀ reached 0.2-0.3. At this time, we add IPTG added at a final title of 2.4 μg/ml and 10¹² M13K07 helper phages (Pharmacia) and shacked again for 5 hours at 37° C. The phages were precipitated twice polyethylene glycol and purified by equilibrium centrifugation in CsCl (2). The quality of the library was tested by titration, the final transducing unit title was 10¹⁰ phages/ml.

Results

Throughout this example, Tyrosine residues that are phosphorylated may be designated “pY”. In the attached sequence listing, this amino acid residue occurs as a normal Tyrosine residue, i.e. “Tyr” or “Y”. In the figures, the phosphotyrosine is referred to as “Z”.

Phage Display on PTP1B

A PTP1B trapping mutant ((12) and Materials and Methods) was used as a positive control to validate the use of a phage display library to study substrate recognition. PTP1B has several advantages: (i) its principal cellular substrate is now well described (9,10), (ii) a reverse alanine scan was performed (19) and gave rise to the determination of an “optimal” substrate consensus sequence and (iii) the crystallographic description of its interaction with a peptide has been precisely mapped (31,32).

After three pannings of the tyrosine-biased library (see Materials and Methods), an enrichment of binding phages was observed. The trapped phages were directly sequenced in order to find a conserved motif (Table III). Then an adapted ELISA assay (see Materials and Methods) was carried out, but this ELISA was not sensitive enough to detect all the trapped clones. Furthermore the success rate of this assay seemed to depend on the PTP tested (see below).

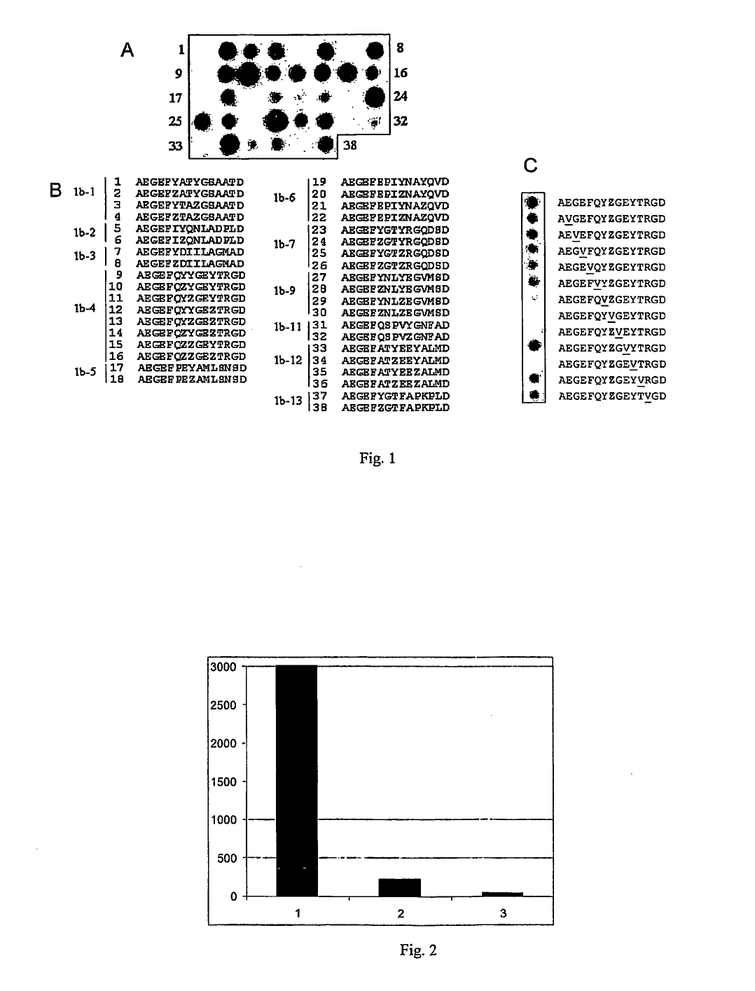

For these experiments it was observed that the capsid of the phage displays two selectable amino acids (E and F at position −2 and −1 of the phosphorylated tyrosine, respectively). This bias can explain why so many positive phages with a tyrosine at the first position of the random displayed sequence were obtained. Nevertheless, some phages like 1b-4 (FIG. 1 and Table III) express an independent conserved motif, where the tyrosine is not preceded by the capsid amino acids. Indeed, it had been demonstrated that a phenolic group in −1 of the substrate tyrosine would stabilise the interaction and, furthermore, place the phosphate in the catalytic pocket (31).

TABLE III
PTP1B selected clones

| Clone | Sequence displayed | | Copy in the pool | ELISA |
|---|---|---|---|---|
| 1b-1 | EFpYATYGSAATDPAK | (SEQ ID NO: 12) | 4× | + |
| 1b-2 | EFIpYQNLADPLDPAK | (SEQ ID NO: 13) | | |
| 1b-3 | EFpYDIILAGMADPAK | (SEQ ID NO: 14) | | |
| 1b-4 | EFQpYZGEYTRGDPAK | (SEQ ID NO: 15) | | |
| 1b-5 | EFPEpYAMLSNSDPAK | (SEQ ID NO: 16) | | |
| 1b-6 | EFEPIpYNAYQVDPAK | (SEQ ID NO: 17) | | |
| 1b-7 | EFpYGTYRGQDSDPAK | (SEQ ID NO: 18) | | + |
| 1b-9 | EFpYNLYEGVMSDPAK | (SEQ ID NO: 19) | | |
| 1b-11 | EFQSPVpYGNFADPAK | (SEQ ID NO: 20) | | |
| 1b-12 | EFATpYEEYALMDPAK | (SEQ ID NO: 21) | | |
| 1b-13 | EFpYGTFAPKPLDPAK | (SEQ ID NO: 22) | | |
| 1b-14 | EFpYGTYRGQDSDPAK | (SEQ ID NO: 23) | 2× | |

Consensus: φpYGXY

It was then tested whether a consensus peptide which had been selected by the trapping mutant would be dephosphorylated by WT PTP1B. By sub-cloning the displayed sequence into a GST expressing vector, the ability of PTP1B to dephosphorylate the sequence that was found to be the most frequent one was tested (1b-1). It was found that the WT form of PTP1B can dephosphorylate the displayed peptide (not shown). As already observed by others, it was noted that the GST-tag was not phosphorylated by Elk kinase in TKB1 cells, meaning that specific dephosphorylation of the displayed peptide was detected.

Finally, in order to confirm the binding of isolated consensus sequences from phages, a SPOT analysis was carried out (Espanel et al., in press). This technique allowed to test 10 sequences (Table III) and check if results comparable to the ELISA could be obtained. Peptides were synthesized on the membrane (see Materials and Methods) with or without a phospho-Tyr (pY), using the SPOT protocol.

Furthermore all possible phosphopeptide combinations were checked for more than one Tyr. All except two sequences (1b-6 and 1b11) were recognized by the PTP1B trapping mutant Interestingly, these two sequences share a PηpYXXφ consensus (X represents any amino acid, n is Tyr or Phe and φ any hydrophobic amino acid). Some sequences with a favourable consensus for PTP1B were not found in ELISA but were confirmed in SPOT (e.g. 1b-9, 1b-4). Double phosphorylated peptides did not seem to react as well as single ones (e.g. 1b-1 spot 4 or 1b-12 spot 36). An interesting exception was SPOT 15 (clone 1b-4): when two phosphorylated Tyr followed each other, it seemed that the binding became more efficient than when two phosphorylated Tyrosines were separated by other amino acids.

As sequence 1b-4 was the best substrate in this assay, it was chosen for a Valine scan (FIG. 1C). In this assay, each position is exchange with a Valine and the binding is tested. Three positions were decisive for the binding on this peptide, namely −1, +1 and +3.

A minimum favourable sequence could be defined around the phospho-Tyr (pY) as

| | |
|---|---|
| EFpYG/ATYG/A | (SEQ ID NO: 4 to 8) |

with a probable preference for a nucleophilic amino acid in +2 (Ser or Thr) and a Tyr in +3 rather than a Phe (e.g. in 1b-13, the signal is weaker even if the rest of the consensus is conserved).

Phage Display on PTP Sap1 and PTP-β

PTP-Sap1 and PTP-β belong to the same subfamily of PTPs (1,2).

A panning of the Tyr-biased library, as done with PTP1B, was performed and a phage enrichment for both PTPs could be observed after three rounds (FIG. 2). Single clones were tested in ELISA. PTP-Sap1 was very efficient in trapping and positive clones could be detected easily (Table IV), but PTP-β was able to trap only three phages out of 30 tested using this method (Table V). The sequences of the Sap-1 clones were reminiscent of the ones of PTP1B (Table II). A Phenyl group at position −1 and +3 (with a preference for Phe) and an invariant Glycine at +1 seem to be the minimum consensus sequence that is recognized. Dephosphorylation assays showed a conserved enzymatic activity for all the clones tested (not shown). PTP-β selected clones were more variable despite the enrichment observed, and those positive in ELISA (Table VI) caryied either a Lys or a Thr in −1 (hydrophobic amino acid). The sequence of the positive clones showed a preference for one of these two amino acids at this position. Furthermore, the phosphatase seems to be less efficient in the catalysis assay, indeed the dephosphorylation of the GST-peptide was weak (not shown) as compared to the same experiment on PTP-Sap1.

TABLE IV
PTP-Sap1 selected clones:

| | | | |
|---|---|---|---|
| Clone | Sequence displayed | Copy in the pool | ELISA |

| | | | | |
|---|---|---|---|---|
| x2 | EFpYGQFpYGPPQDPAK | (SEQ ID NO: 24) | | + |
| x3 | EFpYGAYTSTTADPAK | (SEQ ID NO: 25) | | + |
| x5 | EFpYGAYSNADLDPAK | (SEQ ID NO: 26) | 2X | + |
| x9 | EFpYGTFAQSAEDPAK | (SEQ ID NO: 27) | 3X | + |
| x10 | EFpYGAFGDFTKDPAK | (SEQ ID NO: 28) | | + |
| x41 | EFpYGELGHISQDPAK | (SEQ ID NO: 29) | | + |
| x42 | EFDVpYGSATSMDPAK | (SEQ ID NO: 30) | | + |
| x50 | EFpYGSFFPISQDPAK | (SEQ ID NO: 31) | | − |
| x51 | EFpYGAFGAP. | (SEQ ID NO: 32) | | + |
| x58 | EFpYGPVASDASDPAK | (SEQ ID NO: 33) | | + |

Consensus: FpYGAφ

TABLE V
PTP-beta selected clones:

| | | |
|---|---|---|
| Clone | Sequence displayed | ELISA |

| | | | |
|---|---|---|---|
| B1 | EFLpYQSFSGNVDPAK | (SEQ ID NO: 34) | ++ |
| B27 | EFLpYGSFFRPPDPAK | (SEQ ID NO: 35) | + |
| B32 | EFTpYQTYSPAADPAK | (SEQ ID NO: 36) | + |

Consensus: LpYQ/GSF

TABLE VI

| | Km (mM) | |
|---|---|---|
| Sap-1WT | 2.665 | +/−0.463 |
| Sap-1R88N | 2.67 | +/−0.2295 |

Spot Analysis of the Positive Clones of PTP-Sap1

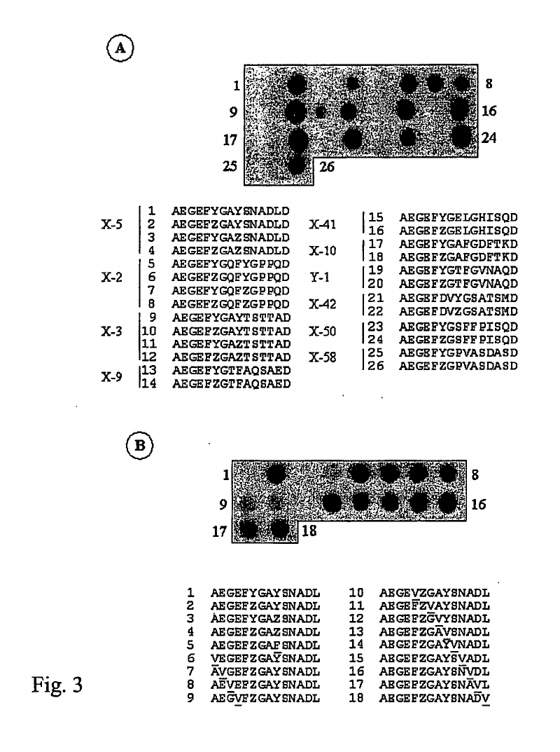

Like for PTP-1B, the positive clones of PTP-Sap1 were tested by SPOT. Almost all Sap-specific sequences were positive in the ELISA test. The best phages shared the following consensus sequence: EFpYGAφ. In the SPOT analysis (FIG. 3A), all sequences were positive, and again the doubly phosphorylated phages gave a weaker signal (e.g. X-5). Furthermore the sequence context for the second Tyr also influenced the interaction. For instance, X-2 gave comparable SPOT results for both phosphorylated Tyrosines, this is probably due to the fact that the second Tyr is also in a favourable environment (FpYG). This example supports the hypothesis that a positive selection for the second Tyr is also possible.

It was then decided to perform a Valine scan in order to describe the necessary amino acids in a favourable sequence (FIG. 3B). X-5 was chosen since it came out twice and its binding in the ELISA assay was the strongest. In this assay, spots 9 to 12 seemed to be critical. When one of these amino acid was changed to a Valine, it dramatically reversed the binding. The Gly in +1 position was also a selective position. It originated from the random sequence and had been selected in all the sequenced clones. In addition, the Ala in +2 was less essential, but small amino acids seemed to be selected at this position on phage display, thus the replacement with a Val does not abrogate the binding. Finally, the phenyl group in +3 present in almost all the selected clones, did not cancel the binding when replaced by a Val. By looking carefully at the sequences of the phages, X-58 has a Val in +3 and was recognized in ELISA, meaning that the Valine scan at this position only exchanges a favourable amino acid (Phe) by a weaker (Val) one. Indeed Valine has been found to be essential at this position for two PTPs, SHP-1 and SHP-2 (not shown).

From this analysis, it could be concluded that there are three selective positions (−2, −1, +1) that can not be replaced by a Val and which seem to be restricted to their structure group. There are probably other positions (+2 and +3) that are more flexible when replaced. Thus the consensus sequence for PTP-Sap1 is EFpYGAFA/G

Changing the Substrate Consensus Sequence by Mutating a Key Residue Within the Catalytic Domain of PTP-Sap1

The sequence identity between the catalytic domains of PTP-β and PTP-SAP-1 is very high (more than 50%). Nevertheless, the selected clones differed in several amino acids: position −1 which is a strict Phe imposed by the capsid sequence for PTP-Sap1 turned out to be a Leu or a Thr for PTP-5. Co-crystallisation of PTP1B with a substrate peptide had been mapped previously (31,32). A precise description of the interaction of Arg47 (catalytic domain numbering, (19,20,31-33)) has been shown. This amino acid is thought to interact with the Phe −1 in an “ideal” peptide substrate. A simple alignment of this region showed that the corresponding amino acid at this position for PTP-Sap1 is an Arg, Arg88, and for PTP-β an Asn, Asn101 (FIG. 4). This position could in fact be the key modulator of the affinity for the position −1 in a Tyr-phosphorylated peptide, explaining the similarity between PTP-Sap1 and PTP1B clones.

To test this hypothesis, a PTP-Sap1R88N mutant was created in a WT and in a trapping mutant background. First, the catalytic activity of the mutant was tested against a generic phosphatase substrate (pNPP) in order to be sure that this mutation would not modify the non-specific recognition of a substrate (Table VI).

Then, the trapping mutant was challenged against the Tyr-biased library. After three rounds, an enrichment of the bound phages was observed, this increase in number of clones was phospho-Tyr dependent (a factor between 10³-10⁴). After DNA sequencing, we observed that the PTP-Sap1 R88N trapping mutant could interact with a conserved family of phages, their sequences were different from that of the clones trapped by PTP-Sap1 (Table VII).

TABLE VII

| | |
|---|---|
| | SEQ ID NO in |
| Peptide sequence | sequence listing |

SHP1

| | | |
|---|---|---|
| 1 | EFYAQIPYRPPDPAK | SEQ ID NO: 37 |
| 2 | EFYAQIPYRPPDPAK | SEQ ID NO: 38 |
| 4 | EFPVYATMNGLDPAK | SEQ ID NO: 39 |
| 5 | EFYYAKVGSVQDPAK | SEQ ID NO: 40 |
| 6 | EFYAEVGRSPPDPAK | SEQ ID NO: 41 |
| 7 | EFPLYAHVNPPDPAK | SEQ ID NO: 42 |
| 8 | EFYAEVGRSPPDPAK | SEQ ID NO: 43 |
| 9 | EFYAEVGRSPPDPAK | SEQ ID NO: 44 |
| 10 | EFSEYAHVQSRDPAK | SEQ ID NO: 45 |

SHP2

| | | |
|---|---|---|
| 1 | EFYAAVSKSPPDPAK | SEQ ID NO: 46 |
| 2 | EFYYAKVGSVQDPAK | SEQ ID NO: 47 |
| 3 | EFYAEVGRSPPDPAK | SEQ ID NO: 48 |
| 4 | EFYAEVGRSPPDPAK | SEQ ID NO: 49 |
| 5 | EFYSTIGRGNSDPAK | SEQ ID NO: 50 |
| 6 | EFYAEVGRSPPDPAK | SEQ ID NO: 51 |
| 7 | EFYAEVGRSPPDPAK | SEQ ID NO: 52 |
| 8 | EFYTIIDRTSTDPAK | SEQ ID NO: 53 |
| 10 | EFTYANVNHPPDPAK | SEQ ID NO: 54 |

PTP-beta library 6

| | | |
|---|---|---|
| 1 | ELVYGLYQSATDPAK | SEQ ID NO: 55 |
| 3 | ELLYGIYHPHSDPAK | SEQ ID NO: 56 |
| 4 | ELYGSLYASDSDPAK | SEQ ID NO: 57 |
| 5 | ELVYWLYGSETDPAK | SEQ ID NO: 58 |
| 6 | ELLYGDYNPRSDPAK | SEQ ID NO: 59 |
| 7 | ELGDLYYGVYLDPAK | SEQ ID NO: 60 |
| 8 | ELFYGEYALFQDPAK | SEQ ID NO: 61 |
| 9 | ELYGSLYLIREDPAK | SEQ ID NO: 62 |
| 10 | ELIYANYREVSDPAK | SEQ ID NO: 63 |
| 11 | ELYGSLYYPVTDPAK | SEQ ID NO: 64 |
| 14 | ELLYGVYSQVADPAK | SEQ ID NO: 65 |
| 15 | ELYGSLYASDSDPAK* | SEQ ID NO: 49 |
| 16 | ELIYGIYFGAPDPAK | SEQ ID NO: 66 |

*Sequence 15 is a duplication of PTP-beta sequence 4

The occurrence of a hydrophobic amino acid at position −1 resembles the clones observed in PTP-β pool. Thus PTP-Sap1 R88N has become PTP-β like in its substrate preference. Indeed, since the −1 position was changed, this reinforces the idea that this position strongly interacts during the recognition process with an Arginine or a Lysine (e.g. PTP SHP-1/2) of the catalytic domain. PTP-Sap1 R88N mutant did not strictly select a Leu in −1, this suggests that probably other amino-acids are involved in the selectivity of this position, nevertheless, most of the selected clones carry a hydrophobic amino acid (η) in −1.

Table VI shows the sequences of 8 different PTP-Sap1 R88N clones.

TABLE VIII
PTP-San1 R88N clones:

| | | |
|---|---|---|
| Sm-1 | EFAHLpYGTFREDPAK | (SEQ ID NO: 67) |
| Sm-2 | EFGATpYGVYTSDPAK | (SEQ ID NO: 68) |
| Sm-7 | EFLpYGEIQGTQDPAK | (SEQ ID NO: 69) |
| Sm-8 | EFLpYANVERSSDPAK | (SEQ ID NO: 70) |
| Sm-10 | EFIpYGQILPRSDPAK | (SEQ ID NO: 71) |
| Sm-11 | EFpYGQIGDHLVDPAK | (SEQ ID NO: 72) |
| Sm-12 | EFpYGEYRPRAQDPAK | (SEQ ID NO: 73) |
| Sm-15 | EFIpYGSFHQTADPAK | (SEQ ID NO: 74) |

Consensus: ηpYGXφ.

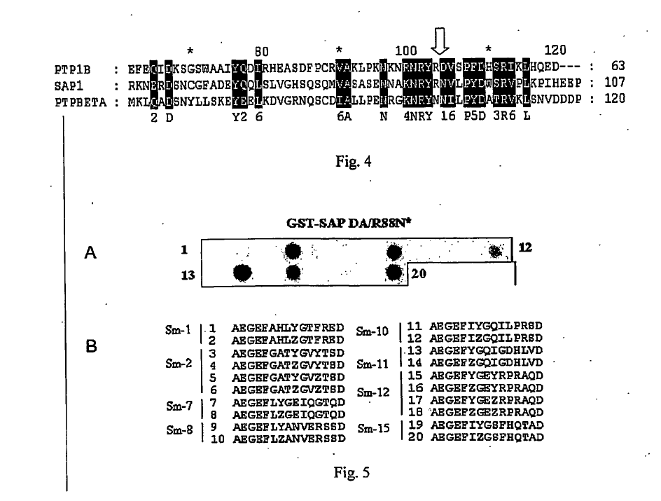

The signal of the ELISA of the pool of clones after every selection round increased, confirming that the positive clone number had been growing during rounds (not shown). Therefore, it was decided to directly challenge our mutant's trapped sequence on a SPOT membrane to test how the minimal consensus was affected. As shown in FIG. 5, the affinity of PTP-Sap1 R88N had changed. Phe is still accepted at position −1, but the substrate recognition has became more flexible: Ile, Leu and Thr can also be recognized in the SPOT assay. Only one clone (sm-8) is not recognized here. The motif preferred by catalytic R88N mutant is:

ηpYGXφ.

Taken together, these data provide for a PTP point mutant study combining phage display to select the clones and SPOT analysis to discover the interacting amino acids from the catalytic domain to the substrate.

Table IX summarizes the results obtained in the frame of the above-presented study. All sequences have a phosphotyrosine (pY) in position 0.

Sap1:

−2: 50% E or 50% L/V (hydrophobic)

−1: 100% hydrophobic (77% I/L)

+1: 100% G

+2: 36% A, 29% T, 14% S and 21% others

+3: hydrophobic, 85% phenolic goup (20% Y and 65% F)

+4: 58% A or G

[symbol] Consensus: ELpYGSYYA (SEQ ID NO: 1)

[symbol] Example: EFpYGAFA/G (Km=7.0 uM) (SEQ ID NO: 2, 3)

[symbol] Example: AEGELpYGSLYA (Km=7.6 uM) (SEQ ID NO: 4)

PTP1B:

−2: 60% E and 20% P

−1: 100% hydrophobic (with 60% F)

+1: 66% G/A

+2: 47% T

+3: 100% hydrophobic, with 80% phenolic (67% Y and 13% F); 20% I/L

+4: 53% G/A

[symbol] Consensus: EFpYA/GTYG/A (SEQ ID NO: 5-8)

PTP-β

−3: 50% acidic (E or D)

−2: 62% L, 13% E

−1: 100% hydrophobic (62% L)

+1: 100% A (23%) or G (77%)

+2: 38% S

+3: 62% Y, 23% L and 15% acidic

+4: 31% phenolic (Y or F)

[symbol] Consensus: ELLpYGSYY (SEQ ID NO: 9)

SHP1:

−2: 56% E, 22% P

−1: 89% hydrophobic (56% F, rest is Y or L)

+1: 100% A

+2: 33% E, 22% Q or H

+3: 89% hydrophobic (67% V, 22% 1)

+4: 44% G [symbol] Consensus: EFpYAEVG (SEQ ID NO: 10)

SHP2:

−2: 77% E and 27% F

−1: 100% hydrophobic with 90% phenolic (77% F)

+1: 77% A

+2: 44% E

+3: 77% V 23% I

+4: 66% G

+5: 66% R

[symbol] Consensus: EFpYAEVGR (SEQ ID NO: 11)

Summary and Discussion

A library of random peptides expressed on a phage capsid was used in order to study the catalytic domain specificity of three PTPs. An enrichment of a pool of trapped phages was observed after each round of panning, and single phages could be trapped by the PTP trapping mutant. When the same sequence was displayed as a GST fusion protein, the wild type PTP could dephosphorylate it.

The combination of phage display and SPOT technology has thus resulted in the definition of the principal amino acids involved in the recognition of a substrate by these PTPs.

Finally, it was shown that amino acids predicted to be directly involved in the substrate recognition could in fact be confirmed when mutated, since they change the preferred substrate sequence.

SHP1 and SHP2

Using procedures outlined above, optimized substrate sequences for SHP1 and SHP2 (Table VIII) could also be obtained. The two PTPs have related catalytic domains, and their optimal sequences, EFZAEVG (SEQ ID NO: 10) and EFZAEVGR (SEQ ID NO: 11) are strongly related as well, but distinct from other PTP recognition motifs.

Rescreening of PTP-β on an Unbiased Library

As mentioned earlier, many of the motifs that were found to incorporate the two capsid-encoded amino acids. Therefore a second library was generated (“library 6”, see experimental procedures) in which the random sequence was presented in a different context. Codons encoding a tyrosine were included the middle of the random sequence. Using this additional study, the motif was extended to ELLZGSYY (SEQ ID NO: 9). This independent experiment further showed that the procedure is reproducible in identifying optimal PTP recognition sequences.

Using this technique, ideal substrates were defined for the following protein tyrosine phosphatases: PTP1B, Sap1, PTP-β, SHP1 and SHP2. These peptides can serve as highly specific inhibitors for the respective phosphatase substrates, and are therefore useful in those diseases, in which inhibition of the respective phosphatase is required.

### REFERENCES

1. Hooft van Huijsduijnen, R. (1998) Gene 225, 1-8
2. Andersen, J. N., Mortensen, O. H., Peters, G. H., Drake, P. G., Iversen, L. F., Olsen, O. H., Jansen, P. G., Andersen, H. S., Tonks, N. K., and Moller, N. P. H. (2001) Molecular & Cellular Biology 21, 7117-7136
3. Venter, J. C. e. a. (2001) Science 291, 1304-1351.
4. Lander, E. S. e. a. (2001) Nature 409, 860-921.
5. Zhang, Z. Y. (2001) Current Opinion in Chemical Biology 5, 416-423
6. Goldstein, B. J., Ahmad, F., Ding, W., Li, P. M., and Zhang, W. R. (1998) Mol. Cell. Biochem. 182, 91-99
7. Espanel, X., Wälchli, S., Pescini Gobert, R., El Alama, M., Curchod, M.-L., Gullu-Isler, N., and Hooft van Huijsduijnen, R. (2001) Endocrine 15, 19-28
8. Wälchli, S., Curchod, M.-L., Pescini Gobert, R., Arkinstall, S., and Hooft van Huijsduijnen, R. (2000) J. Biol. Chem. 275, 9792-9796
9. Elchebly, M., Payette, P., Michaliszyn, E., Cromlish, W., Collins, S., Loy, A. L., Normandin, D., Cheng, A., Himms-Hagen, J., Chan, C. C., Ramachandran, C., Gresser, M. J., Tremblay, M. L., and Kennedy, B. P. (1999) Science 283, 1544-1548
10. Klaman, L. D., Boss, O., Peroni, O. D., Kim, J. K., Martino, J. L., Zabolotny, J. M., Moghal, N., Lubkin, M., Kim, Y. B., Sharpe, A. H., Stricker-Krongrad, A., Shulman, G. I., Neel, B. G., and Kahn, B. B. (2000) Mol. Cell. Biol. 20, 5479-5489
11. Larsen, S. D., Barf, T., Liljebris, C., May, P. D., Ogg, D., O'Sullivan, T. J., Palazuk, B. J., Schostarez, H. J., Stevens, F. C., and Bleasdale, J. E. (2002) J. Med. Chem. 45, 598-622
12. Flint, A. J., Tiganis, T., Barford, D., and Tonks, N. K. (1997) Proc. Natl. Acad. Sci. U.S.A. 94, 1680-1685
13. Garton, A. J., Flint, A. J., and Tonks, N. K. (1996) Mol. Cell. Biol. 16, 6408-6418
14. Cote, J. F., Charest, A., Wagner, J., and Tremblay, M. L. (1998) Biochemistry 37, 13128-13137
15. Zhang, Z. Y., Thieme-Sefler, A. M., Maclean, D., McNamara, D. J., Dobrusin, E. M., Sawyer, T. K., and Dixon, J. E. (1993) Proc. Natl. Acad. Sci. U.S.A. 90, 4446-4450
16. Wu, L., Buist, A., den Hertog, J., and Zhang, Z. Y. (1997) J. Biol. Chem. 272, 6994-7002
17. Huyer, G., Kelly, J., Moffat, J., Zamboni, R., Jia, Z., Gresser, M. J., and Ramachandran, C. (1998) Anal Biochem 258, 19-30
18. Pellegrini, M. C., Liang, H., Mandiyan, S., Wang, K., Yuryev, A., Vlattas, I., Sytwu, T., Li, Y. C., and Wennogle, L. P. (1998) Biochemistry 37, 15598-15606
19. Vetter, S. W., Keng, Y. F., Lawrence, D. S., and Zhang, Z. Y. (2000) J Biol Chem 275, 2265-2268
20. Asante-Appiah, E., Ball, K., Bateman, K., Skorey, K., Friesen, R., Desponts, C., Payette, P., Bayly, C., Zamboni, R., Scapin, G., Ramachandran, C., and Kennedy, B. P. (2001) J. Biol. Chem. 276, 26036-26043
21. Schmitz, R., Baumann, G., and Gram, H. (1996) J Mol Biol 260, 664-677
22. Dente, L., Vetriani, C., Zucconi, A., Pelicci, G., Lanfrancone, L., Pelicci, P. G., and Cesareni, G. (1997) J Mol Biol 269, 694-703
23. Deng, S. J., Liu, W., Simmons, C. A., Moore, J. T., and Tian, G. (2001) Comb Chem High Throughput Screen 4, 525-533
24. Kay, B. K., Winter J., McCafferty J. (ed) (1996) Phage Display of Peptides and Proteins, A Laboratory Manual, Academic Press, INC.
25. Cochrane, D., Webster, C., Masih, G., and McCafferty, J. (2000) J Mol Biol 297, 89-97
26. Felici, F., Castagnoli, L., Musacchio, A., Jappelli, R., and Cesareni, G. (1991) J Mol Biol 222, 301-310
27. Walchli, S., Curchod, M. L., Gobert, R. P., Arkinstall, S., and Hooft van Huijsduijnen, R. (2000) J. Biol. Chem. 275, 9792-9796
28. Espanel, X., and Sudol, M. (2001) J Biol Chem 276, 14514-14523
29. Terry, T. D., Malik, P., and Perham, R. N. (1997) Biol Chem 378, 523-530
30. Murthy, K. K., Ekiel, I., Shen, S. H., and Banville, D. (1999) Biotechniques 26, 142-149
31. Sarmiento, M., Puius, Y. A., Vetter, S. W., Keng, Y. F., Wu, L., Zhao, Y., Lawrence, D. S., Almo, S. C., and Zhang, Z. Y. (2000) Biochemistry 39, 8171-8179
32. Salmeen, A., Andersen, J. N., Myers, M. P., Tonks, N. K., and Barford, D. (2000) Molecular Cell 6, 1401-1412
33. Zhang, Y. L., Yao, Z. J., Sarmiento, M., Wu, L., Burke, T. R., Jr., and Zhang, Z. Y. (2000) J. Biol. Chem. 275, 34205-34212
34. Kole, H. K., Garant, M. J., Kole, S., and Bernier, M. (1996) J. Biol. Chem. 271, 14302-14307
35. Bliska, Jb, Guan, K I, Dixon, Jefalkow, S: Tyrosine phosphate hydrolysis of host proteins by an essential Yersinia virulence determinant. Proc. Natl. Acad. Sci. USA (1991) 88(4):1187-1191.
36. Devinney, I, Steele-Mortimer, Ifinlay, BBb: Phosphatases and kinases delivered to the host cell by bacterial pathogens. Trends Microbiol. (2000) 8(1):29-33.
37. Odenbreit, S, Puls, J, Sedlmaier, B, Gerland, E, Fischer, Whaas, R: Translocation of Helicobacter pylori CagA into gastric epithelial cells by type IV secretion. Science (2000) 287(5457):1497-1500.
38. Higashi, H, Tsutsumi, R, Muto, S Et Al.: SHP-2 tyrosine phosphatase as an intracellular target of Helicobacter pylori CagA protein. Science (2002) 295(5555):683-686.
39. Murli, S, Watson, Rogalan, J E: Role of tyrosine kinases and the tyrosine phosphatase SptP in the interaction of Salmonella with host cells. Cell Microbiol (2001) 3(12):795-810.
40. Pathak, Mkyi, T: Sodium stibogluconate is a potent inhibitor of protein tyrosine phosphatases and augments cytokine responses in hemopoietic cell lines. J. Immunol. (2001) 167(6):3391-3397.
41. Marcus, Bc, Wyble, Cw, Hynes, Klgewertz, B I: Cytokine-induced increases in endothelial permeability occur after adhesion molecule expression. Surgery (1996) 120(2):411-416; discussion 416-417.
42. Jain, Rkmunn, L L: Leaky vessels? Call Ang1! Nat. Med. (2000) 6(2):131-132.
43. Fachinger, G, Deutsch, Urisau, W: Functional interaction of vascular endothelial-protein-tyrosine phosphatase with the angiopoietin receptor Tie-2. Oncogene (1999) 18(43):5948-5953.
44. Noguchi, T., Tsuda, M., Takeda, H., Takada, T., Inagaki, K., Yamao, T., Fukunaga, K., Matozaki, T., and Kasuga, M. 2001. Inhibition of cell growth and spreading by stomach cancer-associated protein-tyrosine phosphatase-1 (SAP-1) through dephosphorylation of p130cas. J. Biol. Chem. 276: 15216-15224.
45. Matozaki, T., and Kasuga, M. 1996. Roles of protein-tyrosine phosphatases in growth factor signalling. Cell. Signal. 8: 13-19.
46. Hooft van Huijsduijnen, R. 1998. Protein Tyrosine Phosphatases: Counting the Trees in the Forest. Gene 225: 1-8.
47. Andersen, J. N., Mortensen, O. H., Peters, G. H., Drake, P. G., Iversen, L. F., Olsen, O. H., Jansen, P. G., Andersen, H. S., Tonks, N. K., and Moller, N. P. 2001. Structural and evolutionary relationships among protein tyrosine phosphatase domains. Mol. Cell. Biol. 21: 7117-7136.
48. Seo, Y., Matozaki, T., Tsuda, M., Hayashi, Y., Itoh, H., and Kasuga, M. 1997. Overexpression of SAP-1, a transmembrane-type protein tyrosine phosphatase, in human colorectal cancers. BioChem. BioPhys. Res. Commun. 231: 705-711.
49. Hooft van Huijsduijnen R, Bombrun A and Swinnen D, Selecting Protein Tyrosine Phosphatases as Drug Targets, Drug Discovery Today, in press
50. Burke T R Jr, Ye B, Yan X, Wang S, Jia Z, Chen L, Zhang Z Y, Barford D. Biochemistry 1996 Dec. 17;35(50):15989-96
51. Groves M R, Yao Z J, Roller P P, Burke T R Jr, Barford D. Biochemistry 1998 Dec. 22;37(51):17773-83
52. Bordo D and Argos P, J Mol Biol 1991, 217: 721-9
53. Rogov S I and Nekrasov A N, Protein Eng 2001, 14: 459-463
54. Murphy L R et al., Protein Eng. 2000, 13:149-52
55. Lindgren et al., TIPS 21, 2000. 99-103
56. Schwarze and Dowdy, TiPS 21, 2000, 45-48
57. Friden et al., Science 1993, 259: 373-377
58. Ranney D F, Biochem Pharmacol 2000, 59: 105-14
59. Ranney D F, Biochem Pharmacol 2000, 59: 105-14
60. Hruby V J and Balse P M, Curr Med Chem 2000, 7:945-70
61. Golebiowski A et al., Curr Opin Drug Discov Devel 2001, 4: 428-34
62. Kim H O and Kahn M, Comb Chem High Throughput Screen 2000; 3: 167-8
63. Cochran A G, Curr Opin Chem Biol 2001, 5(6): 654-659
64. Dougherty D A, Curr Opin Chem Biol 2000, 4: 645-52).
65. Bjorge, J. D., Pang, A. and Fujita, D. J. (2000). “Identification of protein-tyrosine phosphatase 1B as the major tyrosine phosphatase activity capable of dephosphorylating and activating c-Src in several human breast cancer cell lines.” J. Biol. Chem. 275(52): 41439-46.
66. Wiener, J. R., Hurteau, J. A., Kerns, B. J., Whitaker, R. S., Conaway, M. R., Berchuck, A. and Bast, R. C., Jr. (1994). “Overexpression of the tyrosine phosphatase PTP1B is associated with human ovarian carcinomas.” Am. J. Obstet. Gynecol. 170(4): 1177-83.
67. Buckley, D. A., Cheng, A., Kiely, P. A., Tremblay, M. L. and O'Connor, R. (2002). “Regulation of insulin-like growth factor type I (IGF-1) receptor kinase activity by protein tyrosine phosphatase 1B (PTP-1B) and enhanced IGF-I-mediated suppression of apoptosis and motility in PTP-1B-deficient fibroblasts.” Mol. Cell. Biol. 22(7): 1998-2010.
68. Wang Peng et al., (2002). Screening combinatorial libraries by mass spectrometry. 2. Identification of optimal substrates of protein tyrosine phosphatase SHP-1. Biochemistry 41(19), 6202-6210.

1.-25. (canceled)

26. A phosphopeptide comprising an amino acid consensus sequence;
wherein said amino acid consensus sequence comprises amino acid positions −2, −1, 0, +1, +2, +3 and +4;
wherein said amino acid positions are defined by reference to amino acid position 0 which is a phosphorylated tyrosine (Y) residue;
wherein the amino acid at position −2 is selected from the group consisting of E, L and V;
wherein the amino acid at position −1 is a hydrophobic amino acid;
wherein the amino acid at position +1 is G;
wherein the amino acid at position +2 is selected from the group consisting of A, T and S;
wherein the amino acid at position +3 is selected from the group consisting of a hydrophobic amino acid and a phenolic amino acid; and
wherein the amino acid at position +4 is selected from the group consisting of A and G.

27. The phosphopeptide of claim 26, wherein the hydrophobic amino acid at position −1 is selected from the group consisting of I and L.

28. The phosphopeptide of claim 26, wherein the amino acid at position +3 is selected from the group consisting of F and Y.

29. The phosphopeptide of claim 26, wherein said phosphopeptide comprises an amino acid sequence selected from the group consisting of ELYGSYYA (SEQ ID NO: 1), EFYGAFA (SEQ ID NO: 2), EFYGAFG (SEQ ID NO: 3), and AEGELYGSLYA (SEQ ID NO: 4).

30. A phosphopeptide comprising an amino acid consensus sequence;
wherein said amino acid consensus sequence comprises amino acid positions −2, −1, 0, +1, +2, +3 and +4;
wherein said amino acid positions are defined by reference to amino acid position 0 which is a phosphorylated tyrosine (Y) residue;
wherein the amino acid at position −2 is selected from the group consisting of E and P;
wherein the amino acid at position −1 is a hydrophobic amino acid;
wherein the amino acid at position +1 is selected from the group consisting of G and A;
wherein the amino acid at position +2 is T;
wherein the amino acid at position +3 is a hydrophobic amino acid; and
wherein the amino acid at position +4 is selected from the group consisting of G and A.

31. The phosphopeptide of claim 30, wherein the hydrophobic amino acid at position −1 is F.

32. The phosphopeptide of claim 30, wherein the hydrophogic amino acid at position +3 is selected from the group consisting of Y, F, I and L.

33. The phosphopeptide of claim 30, wherein said phosphopeptide comprises an amino acid sequence selected from the group consisting of EFYATYG (SEQ ID NO: 5), EFYGTYG (SEQ ID NO: 6), EFYATYA (SEQ ID NO: 7) and EFYGTYA (SEQ ID NO: 8).

34. A phosphopeptide comprising an amino acid consensus sequence;
wherein said amino acid consensus sequence comprises amino acid positions −3, −2, −1, 0, +1, +2, +3 and +4;
wherein said amino acid positions are defined by reference to amino acid position 0 which is a phosphorylated tyrosine (Y) residue;
wherein the amino acid at position −3 is an acidic amino acid;
wherein the amino acid at position −2 is selected from the group consisting of L and E;
wherein the amino acid at position −1 is a hydrophobic amino acid;
wherein the amino acid at position +1 is selected from the group consisting of G and A;
wherein the amino acid at position +2 is S;
wherein the amino acid at position +3 is a selected from the group consisting of Y, L and acidic amino acids; and
wherein the amino acid at position +4 is a phenolic amino acid.

35. The phosphopeptide of claim 34, wherein the acidic amino acid at position −3 is selected from the group consisting of E and D.

36. The phosphopeptide of claim 34, wherein the hydrophobic amino acid at position −1 is L.

37. The phosphopeptide of claim 34, wherein the phenolic amino acid at position −4 is selected from the group consisting of Y and F.

38. The phosphopeptide of claim 34, wherein said phosphopeptide comprises the amino acid sequence ELLYGSYY (SEQ ID NO: 9).

39. A phosphopeptide comprising an amino acid consensus sequence;
wherein said amino acid consensus sequence comprises amino acid positions −3, −2, −1, 0, +1, +2, +3 and +4;
wherein said amino acid positions are defined by reference to amino acid position 0 which is a phosphorylated tyrosine (Y) residue;
wherein the amino acid at position −2 is selected from the group consisting of E and P;
wherein the amino acid at position −1 is a hydrophobic amino acid;
wherein the amino acid at position +1 is A;
wherein the amino acid at position +2 is selected from the group consisting of E, Q and H;
wherein the amino acid at position +3 is a hydrophobic amino acid; and
wherein the amino acid at position +4 is G.

40. The phosphopeptide of claim 39, wherein the hydrophobic amino acid at position −1 is selected from the group consisting of F, Y and L.

41. The phosphopeptide of claim 39, wherein the hydrophobic amino acid at position +3 is selected from the group consisting of V and I.

42. The phosphopeptide of claim 39, wherein said phosphopeptide comprises the amino acid sequence EFYAEVG (SEQ ID NO: 10).

43. A phosphopeptide comprising an amino acid consensus sequence;
wherein said amino acid consensus sequence comprises amino acid positions −3, −2, −1, 0, +1, +2, +3, +4 and +5;
wherein said amino acid positions are defined by reference to amino acid position 0 which is a phosphorylated tyrosine (Y) residue;
wherein the amino acid at position −2 is selected from the group consisting of E and F;
wherein the amino acid at position −1 is a hydrophobic amino acid;
wherein the amino acid at position +1 is A;
wherein the amino acid at position +2 is E;
wherein the amino acid at position +3 is a selected from the group consisting of V and I;
wherein the amino acid at position +4 is G; and
wherein the amino acid at position +5 is R.

44. The phosphopeptide of claim 43, wherein the hydrophobic amino acid at position −1 is a phenolic amino acid.

45. The phosphopeptide of claim 43, wherein the hydrophobic amino acid at position −1 is F.

46. The phosphopeptide of claim 43, wherein said phosphopeptide comprises the amino acid sequence EFYAEVGR (SEQ ID NO: 11).

47. (canceled)

48. A peptidomimetic or non-peptide mimetic designed on the basis of the sequence and/or the structure selected from the group consisting of:
(a) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E, L and V; wherein the amino acid at position −1 is a hydrophobic amino acid; wherein the amino acid at position +1 is G; wherein the amino acid at position +2 is selected from the group consisting of A, T and S; wherein the amino acid at position +3 is selected from the group consisting of a hydrophobic amino acid and a phenolic amino acid; and wherein the amino acid at position +4 is selected from the group consisting of A and G;
(b) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is selected from the group consisting of G and A; wherein the amino acid at position +2 is T; wherein the amino acid at position +3 is a hydrophobic amino acid; and wherein the amino acid at position +4 is selected from the group consisting of G and A;
(c) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −3 is an acidic amino acid; wherein the amino acid at position −2 is selected from the group consisting of L and E; wherein the amino acid at position −1 is a hydrophobic amino acid; wherein the amino acid at position +1 is selected from the group consisting of G and A; wherein the amino acid at position +2 is S; wherein the amino acid at position +3 is a selected from the group consisting of Y, L and acidic amino acids; and wherein the amino acid at position +4 is a phenolic amino acid;
(d) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is A; wherein the amino acid at position +2 is selected from the group consisting of E, Q and H: wherein the amino acid at position +3 is a hydrophobic amino acid; and wherein the amino acid at position +4 is G; and
(e) a phosphopeptide comprising an amino acid consensus sequence; wherein said amino acid consensus sequence comprises amino acid positions −3, −2, −1, 0, +1, +2, +3, +4 and +5; wherein said amino acid positions are defined by reference to amino acid position 0 which is a phosphorylated tyrosine (Y) residue; wherein the amino acid at position −2 is selected from the group consisting of E and F; wherein the amino acid at position −1 is a hydrophobic amino acid; wherein the amino acid at position +1 is A; wherein the amino acid at position +2 is E; wherein the amino acid at position +3 is a selected from the group consisting of V and I; wherein the amino acid at position +4 is G: and wherein the amino acid at position +5 is R;
wherein said peptidomimetic or non-peptide mimetic does not comprise the amino acid sequence RNNEFYA (SEQ ID NO:75), and wherein Y is a phosphorylated tyrosine residue.

49. A functional derivative of a phosphopeptide selected from the group consisting of:
(a) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E, L and V; wherein the amino acid at position −1 is a hydrophobic amino acid; wherein the amino acid at position +1 is G; wherein the amino acid at position +2 is selected from the group consisting of A, T and S; wherein the amino acid at position +3 is selected from the group consisting of a hydrophobic amino acid and a phenolic amino acid; and wherein the amino acid at position +4 is selected from the group consisting of A and G;
(b) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is selected from the group consisting of G and A; wherein the amino acid at position +2 is T; wherein the amino acid at position +3 is a hydrophobic amino acid; and wherein the amino acid at position +4 is selected from the group consisting of G and A;
(c) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −3 is an acidic amino acid; wherein the amino acid at position −2 is selected from the group consisting of L and E; wherein the amino acid at position −1 is a hydrophobic amino acid; wherein the amino acid at position +1 is selected from the group consisting of G and A; wherein the amino acid at position +2 is S; wherein the amino acid at position +3 is a selected from the group consisting of Y, L and acidic amino acids; and wherein the amino acid at position +4 is a phenolic amino acid;
(d) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is A; wherein the amino acid at position +2 is selected from the group consisting of E, O and H; wherein the amino acid at position +3 is a hydrophobic amino acid; and wherein the amino acid at position +4 is G; and
(e) a phosphopeptide comprising an amino acid consensus sequence; wherein said amino acid consensus sequence comprises amino acid positions −3, −2, −1, 0, +1, +2, +3, +4 and +5; wherein said amino acid positions are defined by reference to amino acid position 0 which is a phosphorylated tyrosine (Y) residue; wherein the amino acid at position −2 is selected from the group consisting of E and F; wherein the amino acid at position −1 is a hydrophobic amino acid; wherein the amino acid at position +1 is A; wherein the amino acid at position +2 is E; wherein the amino acid at position +3 is a selected from the group consisting of V and I; wherein the amino acid at position +4 is G; and wherein the amino acid at position +5 is R;
comprising at least one moiety attached to said phosphopeptide, wherein the functional derivative does not comprise the amino acid sequence RNNEFYA (SEQ ID NO:75), and wherein Y is a phosphorylated tyrosine residue.

50. (canceled)

51. (canceled)

52. A method of treating or preventing a PTP mediated disease comprising administering to a patient in need thereof a pharmaceutically effective amount of a phosphopeptide selected from the group consisting of:
(a) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phoshorylated tyrosine (Y) residue; wherein the amino acid at position −2 is selected from the group consisting of E, L and V; wherein the amino acid at position −1 is a hydrophobic amino acid; wherein the amino acid at position +1 is G; wherein the amino acid at position +2 is selected from the group consisting of A, T and S; wherein the amino acid at position +3 is selected from the group consisting of a hydrophobic amino acid and a phenolic amino acid; and wherein the amino acid at position +4 is selected from the group consisting of A and G;
(b) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is selected from the group consisting of G and A; wherein the amino acid at position +2 is T; wherein the amino acid at position +3 is a hydrophobic amino acid; and wherein the amino acid at position +4 is selected from the group consisting of G and A;
(c) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −3 is an acidic amino acid; wherein the amino acid at position −2 is selected from the group consisting of L and E; wherein the amino acid at position −1 is a hydrophobic amino acid; wherein the amino acid at position +1 is selected from the group consisting of G and A; wherein the amino acid at position +2 is S; wherein the amino acid at position +3 is a selected from the group consisting of Y, L and acidic amino acids; and wherein the amino acid at position +4 is a phenolic amino acid;
(d) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is A; wherein the amino acid at position +2 is selected from the group consisting of E, Q and H; wherein the amino acid at position +3 is a hydrophobic amino acid; and wherein the amino acid at position +4 is G; and
(e) a phosphopeptide comprising an amino acid consensus sequence; wherein said amino acid consensus sequence comprises amino acid positions −3, −2, −1, 0, +1, +2, +3, +4 and +5; wherein said amino acid positions are defined by reference to amino acid position 0 which is a phosphorylated tyrosine (Y) residue; wherein the amino acid at position −2 is selected from the group consisting of E and F; wherein the amino acid at position −1 is a hydrophobic amino acid; wherein the amino acid at position +1 is A; wherein the amino acid at position +2 is E; wherein the amino acid at position +3 is a selected from the group consisting of V and I; wherein the amino acid at position +4 is G; and wherein the amino acid at position +5 is R;
or a peptidomimetic, a non-peptide mimetic or a functional derivative of said phosphopeptide.

53. The method of claim 52, wherein said disease is cancer and wherein said phosphopeptide comprises an amino acid consensus sequence comprising amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E, L and V; wherein the amino acid at position −1 is a hydrophobic amino acid; wherein the amino acid at position +1 is G; wherein the amino acid at position +2 is selected from the group consisting of A, T and S; wherein the amino acid at position +3 is selected from the group consisting of a hydrophobic amino acid and a phenolic amino acid; and wherein the amino acid at position +4 is selected from the group consisting of A and G; or a peptidomimetic, a non-peptide mimetic or a functional derivative of said phosphopeptide.

54. (canceled)

55. The method of claim 52, wherein said disease is diabetes and wherein said phosphopeptide comprises an amino acid consensus sequence comprising amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is selected from the group consisting of G and A; wherein the amino acid at position +2 is T; wherein the amino acid at position +3 is a hydrophobic amino acid; and wherein the amino acid at position +4 is selected from the group consisting of G and A; or a peptidomimetic, a non-peptide mimetic or a functional derivative of said phosphopeptide.

56. The method of claim 52, wherein said disease is obesity and wherein said phosphopeptide comprises an amino acid consensus sequence comprising amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is selected from the group consisting of G and A; wherein the amino acid at position +2 is T; wherein the amino acid at position +3 is a hydrophobic amino acid; and wherein the amino acid at position +4 is selected from the group consisting of G and A; or a peptidomimetic, a non-peptide mimetic or a functional derivative of said phosphopeptide.

57. A method of suppressing appetite, comprising administering to a subject in need thereof a therapeutically effective amount of a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is selected from the group consisting of G and A; wherein the amino acid at position +2 is T; wherein the amino acid at position +3 is a hydrophobic amino acid; and wherein the amino acid at position +4 is selected from the group consisting of G and A; or a peptidomimetic, non-peptide mimetic or functional derivative of such phosphopeptide.

58. The method of claim 52, wherein said disease is inflammation and wherein said phosphopeptide comprises an amino acid consensus sequence comprising amino acid positions −3, −2, −1, 0, +1, +2, +3 and +4; wherein said amino acid positions are defined by reference to amino acid position 0 which is a phosphorylated tyrosine (Y) residue; wherein the amino acid at position −3 is an acidic amino acid; wherein the amino acid at position −2 is selected from the group consisting of L and E; wherein the amino acid at position −1 is a hydrophobic amino acid; wherein the amino acid at position +1 is selected from the group consisting of G and A; wherein the amino acid at position +2 is S; wherein the amino acid at position +3 is a selected from the group consisting of Y, L and acidic amino acids; and wherein the amino acid at position +4 is a phenolic amino acid; or a peptidomimetic, a non-peptide mimetic or a functional derivative of said phosphopeptide.

59. The method of claim 52, wherein said disease is multiple sclerosis and wherein said phosphopeptide comprises an amino acid consensus sequence comprising amino acid positions −3, −2, −1, 0, +1, +2, +3 and +4; wherein said amino acid positions are defined by reference to amino acid position 0 which is a phosphorylated tyrosine (Y) residue; wherein the amino acid at position −3 is an acidic amino acid; wherein the amino acid at position −2 is selected from the group consisting of L and E; wherein the amino acid at position −1 is a hydrophobic amino acid; wherein the amino acid at position +1 is selected from the group consisting of G and A; wherein the amino acid at position +2 is S; wherein the amino acid at position +3 is a selected from the group consisting of Y, L and acidic amino acids; and wherein the amino acid at position +4 is a phenolic amino acid; or a peptidomimetic, a non-peptide mimetic or a functional derivative of said phosphopeptide.

60. The method of claim 52, wherein said disease is an angiogenesis-dependent disease and wherein said phosphopeptide comprises an amino acid consensus sequence comprising amino acid positions −3, −2, −1, 0, +1, +2, +3 and +4; wherein said amino acid positions are defined by reference to amino acid position 0 which is a phosphorylated tyrosine (Y) residue; wherein the amino acid at position −3 is an acidic amino acid; wherein the amino acid at position −2 is selected from the group consisting of L and E; wherein the amino acid at position −1 is a hydrophobic amino acid; wherein the amino acid at position +1 is selected from the group consisting of G and A; wherein the amino acid at position +2 is S; wherein the amino acid at position +3 is a selected from the group consisting of Y, L and acidic amino acids; and wherein the amino acid at position +4 is a phenolic amino acid; or a peptidomimetic, a non-peptide mimetic or a functional derivative of said phosphopeptide.

61. (canceled)

62. The method of claim 52, wherein said disease is an infectious disease and wherein said phosphopeptide is selected from the group consisting of:
(a) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is A; wherein the amino acid at position +2 is selected from the group consisting of E, Q and H; wherein the amino acid at position +3 is a hydrophobic amino acid; and wherein the amino acid at position +4 is G; and
(b) a phosphopeptide comprising an amino acid consensus sequence; wherein said amino acid consensus sequence comprises amino acid positions −3, −2, −1, 0, +1, +2, +3, +4 and +5; wherein said amino acid positions are defined by reference to amino acid position 0 which is a phosphorylated tyrosine (Y) residue; wherein the amino acid at position −2 is selected from the group consisting of E and F; wherein the amino acid at position −1 is a hydrophobic amino acid; wherein the amino acid at position +1 is A; wherein the amino acid at position +2 is E; wherein the amino acid at position +3 is a selected from the group consisting of V and I; wherein the amino acid at position +4 is G; and wherein the amino acid at position +5 is R;
or a peptidomimetic, a non-peptide mimetic or a functional derivative of said phosphopeptide.

63. (canceled)

64. A pharmaceutical composition comprising a phosphopeptide selected from the group consisting of:
(a) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E, L and V; wherein the amino acid at position −1 is a hydrophobic amino acid; wherein the amino acid at position +1 is G; wherein the amino acid at position +2 is selected from the group consisting of A, T and S; wherein the amino acid at position +3 is selected from the group consisting of a hydrophobic amino acid and a phenolic amino acid; and wherein the amino acid at position +4 is selected from the group consisting of A and G;
(b) a phosphopeptide comprising an amino acid consensus sequence; wherein said amino acid consensus sequence comprises amino acid positions −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is selected from the group consisting of G and A; wherein the amino acid at position +2 is T; wherein the amino acid at position +3 is a hydrophobic amino acid; and wherein the amino acid at position +4 is selected from the group consisting of G and A;
(c) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue: wherein the amino acid at position −3 is an acidic amino acid; wherein the amino acid at position −2 is selected from the group consisting of L and E; wherein the amino acid at position −1 is a hydrophobic amino acid; wherein the amino acid at position +1 is selected from the group consisting of G and A; wherein the amino acid at position +2 is S; wherein the amino acid at position +3 is a selected from the group consisting of Y, L and acidic amino acids; and wherein the amino acid at position +4 is a phenolic amino acid;
(d) a phosphopeptide comprising an amino acid consensus sequence; wherein said amino acid consensus sequence comprises amino acid positions −3, −2, −1, 0, +1, +2, +3 and +4; wherein said amino acid positions are defined by reference to amino acid position 0 which is a phosphorylated tyrosine (Y) residue; wherein the amino acid at position −2 is selected from the group consisting of E and P; wherein the amino acid at position −1 is a hydrophobic amino acid; wherein the amino acid at position +1 is A; wherein the amino acid at position +2 is selected from the group consisting of E, Q and H; wherein the amino acid at position +3 is a hydrophobic amino acid; and wherein the amino acid at position +4 is G; and
(e) a phosphopeptide comprising an amino acid consensus sequence; wherein said amino acid consensus sequence comprises amino acid positions −3, −2, −1, 0, +1, +2, +3, +4 and +5; wherein said amino acid positions are defined by reference to amino acid position 0 which is a phosphorylated tyrosine (Y) residue; wherein the amino acid at position −2 is selected from the group consisting of E and F; wherein the amino acid at position −1 is a hydrophobic amino acid; wherein the amino acid at position +1 is A; wherein the amino acid at position +2 is E; wherein the amino acid at position +3 is a selected from the group consisting of V and I; wherein the amino acid at position +4 is G; and wherein the amino acid at position +5 is R;
or a peptidomimetic, non-peptide mimetic or functional derivative of said phosphopeptide.

65. (canceled)


SEQUENCE LISTING (84 sequences)

SEQ ID NO: 1 - protein, 8 aa, Artificial Sequence
  Synthetic phosphopeptide
ELYGSYYA

SEQ ID NO: 2 - protein, 7 aa, Artificial Sequence
  Synthetic phosphopeptide
EFYGAFA

SEQ ID NO: 3 - protein, 7 aa, Artificial Sequence
  Synthetic phosphopeptide
EFYGAFG

SEQ ID NO: 4 - protein, 11 aa, Artificial Sequence
  Synthetic phosphopeptide
AEGELYGSLYA

SEQ ID NO: 5 - protein, 7 aa, Artificial Sequence
  Synthetic phosphopeptide
EFYATYG

SEQ ID NO: 6 - protein, 7 aa, Artificial Sequence
  Synthetic phosphopeptide
EFYGTYG

SEQ ID NO: 7 - protein, 7 aa, Artificial Sequence
  Synthetic phosphopeptide
EFYATYA

SEQ ID NO: 8 - protein, 7 aa, Artificial Sequence
  Synthetic phosphopeptide
EFYGTYA

SEQ ID NO: 9 - protein, 8 aa, Artificial Sequence
  Synthetic phosphopeptide
ELLYGSYY

SEQ ID NO: 10 - protein, 7 aa, Artificial Sequence
  Synthetic phosphopeptide
EFYAEVG

SEQ ID NO: 11 - protein, 8 aa, Artificial Sequence
  Synthetic phosphopeptide
EFYAEVGR

SEQ ID NO: 12 - protein, 15 aa, Artificial Sequence
  Peptide Sequence on Phage Display
EFYATYGSAATDPAK

SEQ ID NO: 13 - protein, 15 aa, Artificial Sequence
  Peptide Sequence on Phage Display
EFIYQNLADPLDPAK

SEQ ID NO: 14 - protein, 15 aa, Artificial Sequence
  Peptide Sequence on Phage Display
EFYDIILAGMADPAK

SEQ ID NO: 15 - protein, 15 aa, Artificial Sequence
  Peptide Sequence on Phage Display
EFQYZGEYTRGDPAK

SEQ ID NO: 16 - protein, 15 aa, Artificial Sequence
  Peptide Sequence on Phage Display
EFPEYAMLSNSDPAK

SEQ ID NO: 17 - protein, 15 aa, Artificial Sequence
  Peptide Sequence on Phage Display
EFEPIYNAYQVDPAK

SEQ ID NO: 18 - protein, 15 aa, Artificial Sequence
  Peptide Sequence on Phage Display
EFYGTYRGQDSDPAK

SEQ ID NO: 19 - protein, 15 aa, Artificial Sequence
  Peptide Sequence on Phage Display
EFYNLYEGVMSDPAK

SEQ ID NO: 20 - protein, 15 aa, Artificial Sequence
  Peptide Sequence on Phage Display
EFQSPVYGNFADPAK

SEQ ID NO: 21 - protein, 15 aa, Artificial Sequence
  Peptide Sequence on Phage Display
EFATYEEYALMDPAK

SEQ ID NO: 22 - protein, 15 aa, Artificial Sequence
  Peptide Sequence on Phage Display
EFYGTFAPKPLDPAK

SEQ ID NO: 23 - protein, 15 aa, Artificial Sequence
  Peptide Sequence on Phage Display
EFYGTYRGQDSDPAK

SEQ ID NO: 24 - protein, 15 aa, Artificial Sequence
  Peptide Sequence on Phage Display
EFYGQFYGPPQDPAK

SEQ ID NO: 25 - protein, 15 aa, Artificial Sequence
  Peptide Sequence on Phage Display
EFYGAYTSTTADPAK

SEQ ID NO: 26 - protein, 15 aa, Artificial Sequence
  Peptide Sequence on Phage Display
EFYGAYSNADLDPAK

SEQ ID NO: 27 - protein, 15 aa, Artificial Sequence
  Peptide Sequence on Phage Display
EFYGTFAQSAEDPAK

SEQ ID NO: 28 - protein, 15 aa, Artificial Sequence
  Peptide Sequence on Phage Display
EFYGAFGDFTKDPAK

SEQ ID NO: 29 - protein, 15 aa, Artificial Sequence
  Peptide Sequence on Phage Display
EFYGELGHISQDPAK

SEQ ID NO: 30 - protein, 15 aa, Artificial Sequence
  Peptide Sequence on Phage Display
EFDVYGSATSMDPAK

SEQ ID NO: 31 - protein, 15 aa, Artificial Sequence
  Peptide Sequence on Phage Display
EFYGSFFPISQDPAK

SEQ ID NO: 32 - protein, 9 aa, Artificial Sequence
  Peptide Sequence on Phage Display
EFYGAFGAP

SEQ ID NO: 33 - protein, 15 aa, Artificial Sequence
  Peptide Sequence on Phage Display
EFYGPVASDASDPAK

SEQ ID NO: 34 - protein, 15 aa, Artificial Sequence
  Peptide Sequence on Phage Display
EFLYQSFSGNVDPAK

SEQ ID NO: 35 - protein, 15 aa, Artificial Sequence
  Peptide Sequence on Phage Display
EFLYGSFFRPPDPAK

SEQ ID NO: 36 - protein, 15 aa, Artificial Sequence
  Peptide Sequence on Phage Display
EFTYQTYSPAADPAK

SEQ ID NO: 37 - protein, 15 aa, Artificial Sequence
  Peptide Sequence on Phage Display
EFYAQIPYRPPDPAK

SEQ ID NO: 38 - protein, 15 aa, Artificial Sequence
  Peptide Sequence on Phage Display
EFYAQIPYRPPDPAK

SEQ ID NO: 39 - protein, 15 aa, Artificial Sequence
  Peptide Sequence on Phage Display
EFPVYATMNGLDPAK

SEQ ID NO: 40 - protein, 15 aa, Artificial Sequence
  Peptide Sequence on Phage Display
EFYYAKVGSVQDPAK

SEQ ID NO: 41 - protein, 15 aa, Artificial Sequence
  Peptide Sequence on Phage Display
EFYAEVGRSPPDPAK

SEQ ID NO: 42 - protein, 15 aa, Artificial Sequence
  Peptide Sequence on Phage Display
EFPLYAHVNPPDPAK

SEQ ID NO: 43 - protein, 15 aa, Artificial Sequence
  Peptide Sequence on Phage Display
EFYAEVGRSPPDPAK

SEQ ID NO: 44 - protein, 15 aa, Artificial Sequence
  Peptide Sequence on Phage Display
EFYAEVGRSPPDPAK

SEQ ID NO: 45 - protein, 15 aa, Artificial Sequence
  Peptide Sequence on Phage Display
EFSEYAHVQSRDPAK

SEQ ID NO: 46 - protein, 15 aa, Artificial Sequence
  Peptide Sequence on Phage Display
EFYAAVSKSPPDPAK

SEQ ID NO: 47 - protein, 15 aa, Artificial Sequence
  Peptide Sequence on Phage Display
EFYYAKVGSVQDPAK

SEQ ID NO: 48 - protein, 15 aa, Artificial Sequence
  Peptide Sequence on Phage Display
EFYAEVGRSPPDPAK

SEQ ID NO: 49 - protein, 15 aa, Artificial Sequence
  Peptide Sequence on Phage Display
EFYAEVGRSPPDPAK

SEQ ID NO: 50 - protein, 15 aa, Artificial Sequence
  Peptide Sequence on Phage Display
EFYSTIGRGNSDPAK

SEQ ID NO: 51 - protein, 15 aa, Artificial Sequence
  Peptide Sequence on Phage Display
EFYAEVGRSPPDPAK

SEQ ID NO: 52 - protein, 15 aa, Artificial Sequence
  Peptide Sequence on Phage Display
EFYAEVGRSPPDPAK

SEQ ID NO: 53 - protein, 15 aa, Artificial Sequence
  Peptide Sequence on Phage Display
EFYTIIDRTSTDPAK

SEQ ID NO: 54 - protein, 15 aa, Artificial Sequence
  Peptide Sequence on Phage Display
EFTYANVNHPPDPAK

SEQ ID NO: 55 - protein, 15 aa, Artificial Sequence
  Peptide Sequence on Phage Display
ELVYGLYQSATDPAK

SEQ ID NO: 56 - protein, 15 aa, Artificial Sequence
  Peptide Sequence on Phage Display
ELLYGIYHPHSDPAK

SEQ ID NO: 57 - protein, 15 aa, Artificial Sequence
  Peptide Sequence on Phage Display
ELYGSLYASDSDPAK

SEQ ID NO: 58 - protein, 15 aa, Artificial Sequence
  Peptide Sequence on Phage Display
ELVYWLYGSETDPAK

SEQ ID NO: 59 - protein, 15 aa, Artificial Sequence
  Peptide Sequence on Phage Display
ELLYGDYNPRSDPAK

SEQ ID NO: 60 - protein, 15 aa, Artificial Sequence
  Peptide Sequence on Phage Display
ELGDLYYGVYLDPAK

SEQ ID NO: 61 - protein, 15 aa, Artificial Sequence
  Peptide Sequence on Phage Display
ELFYGEYALFQDPAK

SEQ ID NO: 62 - protein, 15 aa, Artificial Sequence
  Peptide Sequence on Phage Display
ELYGSLYLIREDPAK

SEQ ID NO: 63 - protein, 15 aa, Artificial Sequence
  Peptide Sequence on Phage Display
ELIYANYREVSDPAK

SEQ ID NO: 64 - protein, 15 aa, Artificial Sequence
  Peptide Sequence on Phage Display
ELYGSLYYPVTDPAK

SEQ ID NO: 65 - protein, 15 aa, Artificial Sequence
  Peptide Sequence on Phage Display
ELLYGVYSQVADPAK

SEQ ID NO: 66 - protein, 15 aa, Artificial Sequence
  Peptide Sequence on Phage Display
ELIYGIYFGAPDPAK

SEQ ID NO: 67 - protein, 15 aa, Artificial Sequence
  Peptide Sequence on Phage Display
EFAHLYGTFREDPAK

SEQ ID NO: 68 - protein, 15 aa, Artificial Sequence
  Peptide Sequence on Phage Display
EFGATYGVYTSDPAK

SEQ ID NO: 69 - protein, 15 aa, Artificial Sequence
  Peptide Sequence on Phage Display
EFLYGEIQGTQDPAK

SEQ ID NO: 70 - protein, 15 aa, Artificial Sequence
  Peptide Sequence on Phage Display
EFLYANVERSSDPAK

SEQ ID NO: 71 - protein, 15 aa, Artificial Sequence
  Peptide Sequence on Phage Display
EFIYGQILPRSDPAK

SEQ ID NO: 72 - protein, 15 aa, Artificial Sequence
  Peptide Sequence on Phage Display
EFYGQIGDHLVDPAK

SEQ ID NO: 73 - protein, 15 aa, Artificial Sequence
  Peptide Sequence on Phage Display
EFYGEYRPRAQDPAK

SEQ ID NO: 74 - protein, 15 aa, Artificial Sequence
  Peptide Sequence on Phage Display
EFIYGSFHQTADPAK

SEQ ID NO: 75 - protein, 7 aa, Artificial Sequence
  Synthetic phosphopeptide
RNNEFYA

SEQ ID NO: 76 - protein, 12 aa, Artificial Sequence
  Synthetic peptide
LXXXXYXXXXDP

SEQ ID NO: 77 - DNA, 61 bp, Artificial Sequence
  Synthetic oligonucleotide
atacaattgnnknnknnknnktatnnknnknnknnggatcctacacatgcagctcccggag

SEQ ID NO: 78 - DNA, 20 bp, Artificial Sequence
  Synthetic primer
gtctccgggagctgcatgtg

SEQ ID NO: 79 - DNA, 20 bp, Artificial Sequence
  Synthetic primer
attgtagcggttcttggcgt

SEQ ID NO: 80 - DNA, 40 bp, Artificial Sequence
  Synthetic primer
acgccaagaaccgctacaataatgtgctgccctatgactg

SEQ ID NO: 81 - DNA, 29 bp, Artificial Sequence
  Synthetic primer
tatctcgagtctttcgctgctgagggtga

SEQ ID NO: 82 - DNA, 31 bp, Artificial Sequence
  Synthetic primer
atagcggccgcttgcagggagtcaaaggccg

SEQ ID NO: 83 - DNA, 21 bp, Artificial Sequence
  Synthetic primer
atgaaaaagtctttagtcctc

SEQ ID NO: 84 - DNA, 21 bp, Artificial Sequence
  Synthetic primer
cagcttgataccgatagttgc